(19) United States
(12) Patent Application Publication
(10) Pub. No.: US 2022/0372070 A1
(43) Pub. Date: Nov. 24, 2022

(54) HIGH SALT LOAD CONDITIONING DURING CATION EXCHANGE CHROMATOGRAPHY TO REMOVE PRODUCT-RELATED IMPURITIES

(71) Applicant: AMGEN INC., Thousand Oaks, CA (US)

(72) Inventors: Luis DIAZ, Thousand Oaks, CA (US); Natalia R. GOMEZ, Playa Vista, CA (US)

(73) Assignee: AMGEN INC., Thousand Oaks, CA (US)

(21) Appl. No.: 17/773,886

(22) PCT Filed: Nov. 4, 2020
(86) PCT No.: PCT/US20/58772

§ 371 Date: May 3, 2022

Related U.S. Application Data
(60) Provisional application No. 62/931,863, filed on Nov. 7, 2019.

(51) Int. Cl. C07K 1/18; C07K 16/06

(52) U.S. Cl. CPC C07K 1/18; C07K 16/065

(57) ABSTRACT

The invention relates to high salt load conditioning during cation exchange chromatography for removal of low isoelectric point product-related impurities during manufacture of recombinant multispecific proteins.

This application claims the benefit of U.S. Provisional Application No. 62/931,863, filed Nov. 7, 2019, which is hereby incorporated by reference.

### FIELD OF DISCLOSURE

The present invention relates to the field of biopharmaceutical manufacturing. In particular, the invention relates to methods for removal of low isoelectric point product-related impurities during cation exchange purification operations.

### BACKGROUND

Antibody products are the largest sector of the biopharmaceuticals market and could easily reach hundreds of billions in sales over the next decade. The commercial development of therapeutic monoclonal antibodies began in the 1980s with the approval of the first therapeutic monoclonal antibody and has continued to evolve and expand ever since. While monoclonal antibodies bind a target with high affinity and specificity and have been very successful therapeutic treatments for some indications, they also have limitations. Monoclonal antibodies bind a single target; however many diseases are multifactorial. In cancer immunotherapy, a single target treatment may not be sufficient to destroy or immobilize cancer cells. In addition, some patients receiving monoclonal antibody therapies may fail to respond to treatment or develop drug resistance.

New antibody-like structures such as antibody Fab fragments, Fc-fusion proteins, antibody-drug conjugates, glycol-engineered antibodies, and most especially, bispecific and other multispecific antibody-like structures have been developed to meet these challenges. These antibody-like structures, particularly bispecific antibodies, offer improvements over traditional monoclonal antibody therapeutics and are proving to be effective next-generation of biotherapeutics, with an enormous variety of formats that can be developed to meet even more challenging therapeutic indications.

Bispecific antibodies are the most diverse group of these antibody-like structures with an ever-increasing variety of frameworks to meet the needs of therapeutic indications. These structures combine the binding properties of antibodies with additional molecular properties engineered to suit desired disease indications. Bispecific antibodies are being developed for a variety of indication and uses, such as redirecting immune effector cells to tumor cells for immune response against cancer, blocking signaling pathways, targeting tumor angiogenesis, blocking cytokines, crossing the blood-brain barrier, diagnostic assays, treatment of pathogens, and as delivery agents. (Sedykh et al., Drug Design, Development and Therapy 18(12), 195-208, 2018; Walsh, Nature Biotechnology, 32(10), 992-1000, 2014; Ecker et al., mAbs 7(1), 9-14, 2015; Spiess et al., Mol Immunol 67, 95-106, 2015; Fan et al., J Hematol & Oncology 8:130-143, 2015; Williams et al., Process Design for Bispecific Antibodies, Biopharmaceutical Processing, Development, Design and Implementation of Processes, Jagschies et al., editors, Elsevier Ltd, pages 837-855, 2018).

Development of these multispecific proteins brings new biomanufacturing challenges, particularly with regard to product instability and low expression yields. In particular, purification of multispecific proteins is complicated by the formation of product-related variants, such as homodimers, half-antibodies, aggregates, high and low molecular weight species and the like. These variants share similar structural and physical properties, such as charge, with the multispecific protein of interest, making them difficult to separate from during purification. These product-related impurities lower the yield and activity of the multispecific drug product and impact the robustness of manufacturing processes.

Product-related impurities having similar charge (isoelectric point) to a multispecific protein of interest may co-elute with the multispecific protein during cation exchange chromatography unit operations, complicating purification and lowering yield. It would be beneficial to separate the low pI product-related impurities prior to elution. The invention described herein meets this need by providing high salt load conditioning for removal of these low pI impurities during cation exchange chromatography.

### BRIEF SUMMARY OF THE INVENTION

The invention provides a method of purifying a multispecific protein from a composition comprising the multispecific protein and at least one product-related impurity, the method comprising equilibrating a cation exchange chromatography medium with an equilibration buffer comprising 94-105 mM Sodium chloride; loading the composition on to the cation exchange medium in a load buffer comprises 94-105 mM Sodium chloride; washing the column with at least one wash buffer comprising 94-105 mM Sodium chloride; and eluting the multispecific protein from the cation exchange chromatography medium. In one embodiment the load buffer comprises 94-96 mM Sodium chloride. In a related embodiment the load buffer comprises 96-105 mM Sodium chloride. In a related embodiment the load buffer comprises 94 mM sodium chloride. In a related embodiment the load buffer comprises 96 mM sodium chloride. In a related embodiment the load buffer comprises 105 mM sodium chloride. In one embodiment the load buffer comprises acetate. In a related embodiment the load buffer comprises acetate, pH 4.9-5.1. In a related embodiment the load buffer comprises acetate, pH 5.0±0.05 to 5.0±0.1. In a related embodiment the load buffer comprises 100 mM acetate. In one embodiment the load buffer comprises acetate and 94 mM-105 mM sodium chloride. In one embodiment at least one wash buffer comprises 94-96 mM sodium chloride. In a related embodiment at least one wash buffer comprises 96-105 mM sodium chloride. In a related embodiment at least one wash buffer comprises 94 mM sodium chloride. In a related embodiment at least one wash buffer comprises 96 mM sodium chloride. In a related embodiment at least one wash buffer comprises 105 mM sodium chloride. In one embodiment at least one wash buffer comprises acetate. In one embodiment at least one wash buffer comprises acetate, pH 4.9-5.1. In a related embodiment, at least one wash buffer comprises acetate, pH 5.0±0.05 to 5.0±0.1. In a related embodiment at least one wash buffer comprises 100 mM acetate. In one embodiment at least one wash buffer comprises acetate and 94 mM-105 mM sodium chloride. In one embodiment at least one additional wash buffer comprises 0-26 mM sodium chloride. In one embodiment at least one wash buffer comprises acetate and 94-96 mM sodium chloride, followed by at least one additional wash buffer comprising acetate and 25 mM sodium chloride. In one embodiment at least one wash buffer comprises acetate and 105 mM sodium chloride, followed by at least one additional wash buffer comprising acetate. In one embodiment at least one equilibration buffer comprises 94-96 mM sodium chloride. In a related embodiment at least one equilibration buffer comprises 96-105 mM sodium chloride. In a related embodiment at least one equilibration buffer comprises 94 mM sodium chloride. In a related embodiment at least one equilibration buffer comprises 96 mM sodium chloride. In a related embodiment at least one equilibration buffer comprises 105 mM sodium chloride. In one embodiment the equilibration buffer comprises acetate. In a related embodiment the equilibration buffer comprises acetate, pH 4.9-5.1. In a related embodiment the equilibration buffer comprises acetate, pH 5.0±0.05 to 5.0±0.1. In a related embodiment the equilibration buffer comprises 100 mM acetate. In one embodiment the equilibration buffer comprises acetate and 94 mM-105 mM sodium chloride. In one embodiment the composition is loaded at 10-27 g/L. In one embodiment the composition is loaded at 15-27 g/L. In one embodiment the multispecific protein is eluted from the cation exchange resin by a gradient. In a related embodiment the gradient is linear. In one embodiment the gradient is a salt gradient. In one embodiment the multispecific protein is a bispecific protein. In one embodiment the multispecific protein is a bispecific antibody. In one embodiment is provided a purified, multispecific protein prepared by the method described above. In one embodiment the cation exchange chromatography medium is a resin.

The invention provides a method of reducing low pI impurities in the eluate from cation exchange chromatography, the method comprising equilibrating a cation exchange chromatography medium with an equilibration buffer comprising 94-105 mM Sodium chloride; loading the composition on to the cation exchange medium in a load buffer comprises 94-105 mM Sodium chloride; washing the column with at least one wash buffer comprising 94-105 mM Sodium chloride; and eluting the multispecific protein from the cation exchange chromatography medium; wherein the cation exchange chromatography eluate has reduced low pI impurities compared to the cation exchange chromatography eluate recovered in a corresponding method in which no sodium chloride is used in the equilibration, load, and wash steps. In one embodiment the low pI impurity is a product-related impurity. In one embodiment at least one product-related impurity is a half antibody or 2×, 3×, or 4× light chain-mis-assembly.

The invention provides a method of performing cation exchange chromatography under high salt loading conditions to reduce product-related impurities, the method comprising equilibrating a cation exchange chromatography medium with an equilibration buffer; loading the composition on to the cation exchange medium in a load buffer; washing the column with a first and a second wash buffer; and eluting the multispecific protein from the cation exchange chromatography medium; wherein the equilibration, loading and first wash buffers comprise 94-105 mM Sodium chloride. In one embodiment the second wash buffer comprises 0-26 mM.

The invention provides a method of producing an isolated, purified, recombinant multispecific protein, the method comprising establishing a cell culture in a bioreactor with a host cell expressing the multispecific protein; culturing the host cells to express the multispecific protein; harvesting the recombinant multispecific protein; affinity purifying the harvested recombinant multispecific protein; inactivating virus at low pH in the eluate pool from the affinity purification and neutralizing the pool; equilibrating a cation exchange chromatography medium with an equilibration buffer comprising 94-105 mM Sodium chloride; loading the neutralized affinity purified recombinant multispecific protein on to the equilibrated cation exchange medium in a load buffer comprises 94-105 mM Sodium chloride; washing the cation exchange medium with a wash buffer comprising 94-105 mM Sodium chloride, followed by a second wash buffer comprising 0-26 mM Sodium chloride; eluting the multispecific protein from the cation exchange chromatography medium; loading the cation exchange chromatography eluate comprising the recombinant multispecific protein onto a second chromatography resin in flow through mode; and concentrating the purified recombinant multispecific protein in a formulation buffer. In one embodiment the second chromatography resin is selected from an anion exchange chromatography resin, cation exchange chromatography resin, multi-modal chromatography resin, hydrophobic interaction chromatography resin, and hydroxyapatite chromatography resin. In a related embodiment is provided an isolated, purified, recombinant multispecific protein prepared by the method described above. In a related embodiment is provided a pharmaceutical composition comprising the isolated, purified, recombinant multispecific protein prepared by the method described above.

### BRIEF DESCRIPTION OF THE DRAWINGS

FIG. 1 Shows that impurities (half antibodies and 2× LC) are eluting with the main product, Bi-specific #1.

FIG. 2 Shows that following high salt load conditioning, the low pI impurities flowed through the column between the load and first wash steps. The second wash returned the UV baseline to zero before the elution. During the elution of Bi-specific #1, the elution peak was reduced from four peaks to one peak.

FIG. 3 Shows one elution peak resulting from the high load density, no salt load conditioning, at a steep elution gradient for Bi-specific #2. The low pI product-related impurities did not resolve from the main product under the high load density and are mostly in fractions 1-3.

FIG. 4 Shows that a lower loading density (10 vs 25 g/L) and shallower gradient (8 vs 16 mM/CV) allowed for separation of the main low pI product-impurities into a distinct peak formed by fractions 1-4 fir Bi-specific #2.

FIG. 5 Shows that under high salt loading conditions, there was a reduction in the number of impurity peaks in the elution profile from two peaks to a single peak for Bi-specific #2, with a small shoulder (Fractions 1-3) that still contained some mispaired species (LC1/LC2=2 to 3).

### DETAILED DESCRIPTION OF THE INVENTION

Because there is not much information in the literature relating to downstream processing of multispecific proteins, platforms developed for monoclonal antibodies are often applied (Shulka and Norman, Chapter 26 Downstream Processing of Fc Fusion Proteins, Bispecific Antibodies, and Antibody-Drug Conjugates, in Process Scale Purification of Antibodies Second Edition, Uwe Gottswchalk editor, p 559-594, John Wiley & Sons, 2017). Subjecting multispecific proteins to a cation exchange chromatography (CEX) in bind and elute mode under conditions typical for antibodies and antibody-like proteins, resulted in multiple impurity peaks in the elution profile. These impurities had isoelectric points that were both lower and higher than the main product. The impurities with lower pI eluted prior to the main product, as pre-peaks. This elution profile would not support the development of a robust, sustainable, commercial scale manufacturing process.

The nature of multispecific proteins may make them susceptible to formation of product-related impurities and cell culture conditions may impact the amount of such impurities. These impurities complicate purification and can lower the yield and activity of the desired multispecific protein. It was found that a high salt loading strategy improved the yield of the main product in the CEX eluate pool by removing the lower pI impurities prior to the elution step. By targeting a final sodium chloride concentration of 94-105 mM for the equilibration buffer, the final conditioned load buffer, and the first wash buffer, the lower pI impurities flowed through the column, reducing the number peaks in the elution profile and reducing the amount of product-related impurities in the CEX eluate. A second wash step was also added to ensure complete binding conditions for the desired multispecific protein and to reestablish the UV baseline to zero before the start of the elution, thereby tightening the elution profile, resulting in a much more efficient collection and better quality of the main product. For example, the high salt load conditioning unexpectedly reduced length of the elution for a bi-specific protein from 44 column volumes (CVs) to 20.3 CVs, a savings in time and resources as well as a reduction in CEX eluate pool volume which is highly desirable for process efficiency and robustness in intermediate downstream unit operations. For both bi-specifics, the high salt loading conditions allowed for efficient removal of impurities prior to elution, reducing the amount of product-related impurities in the CEX eluate, as well as simplifying the elution collection criteria, thus providing a more robust manufacturing process.

The invention provides a method of purifying a multispecific protein from a composition comprising the multispecific protein and at least one product-related impurity, the method comprising equilibrating a cation exchange chromatography medium with an equilibration buffer comprising 94-105 mM sodium chloride; loading the composition on to the cation exchange medium in a load buffer comprises 94-105 mM sodium chloride; washing the column with at least one wash buffer comprising 94-105 mM sodium chloride; and eluting the multispecific protein from the cation exchange chromatography medium.

The invention also provides a method of reducing low pI impurities in the eluate from cation exchange chromatography, the method comprising equilibrating a cation exchange chromatography medium with an equilibration buffer comprising 94-105 mM sodium chloride; loading the composition on to the cation exchange medium in a load buffer comprises 94-105 mM sodium chloride; washing the column with at least one wash buffer comprising 94-105 mM sodium chloride; and eluting the multispecific protein from the cation exchange chromatography medium; wherein the cation exchange chromatography eluate has reduced low pI impurities compared to the cation exchange chromatography eluate recovered in a corresponding method in which no sodium chloride is used in the equilibration, load, and wash steps.

The invention also provides a method of performing cation exchange chromatography under high salt loading conditions to reduce product-related impurities, the method comprising equilibrating a cation exchange chromatography medium with an equilibration buffer; loading the composition on to the cation exchange medium in a load buffer; washing the column with a first and second wash buffer; and eluting the multispecific protein from the cation exchange chromatography medium; wherein the equilibration, loading and first wash buffers comprise 94-105 mM sodium chloride.

The invention also provides a method of producing an isolated, purified, recombinant multispecific protein of interest, the method comprising establishing a cell culture in a bioreactor with a host cell expressing the multispecific protein of interest; culturing the host cells to express the multispecific protein; harvesting the recombinant multispecific protein; affinity purifying the harvested recombinant multispecific protein; inactivating virus at low pH in the eluate pool from the affinity purification and neutralizing the pool; equilibrating a cation exchange chromatography medium with an equilibration buffer comprising 94-105 mM sodium chloride; loading the neutralized affinity purified recombinant multispecific protein on to the equilibrated cation exchange medium in a load buffer comprises 94-105 mM sodium chloride; washing the cation exchange medium with a wash buffer comprising 94-105 mM sodium chloride, followed by a second wash buffer comprising 0-26 mM Sodium chloride; eluting the multispecific protein from the cation exchange chromatography medium; and loading the cation exchange chromatography eluate comprising the recombinant multispecific protein onto a second chromatography resin in flow through mode; and concentrating the purified recombinant multispecific protein in a formulation buffer.

In one embodiment the load buffer comprises 94-105 mM sodium chloride. In one embodiment the load buffer comprises 94, 95, 96, 97, 98, 99, 100, 101, 102, 103, 104, or 105 mM sodium chloride. In one embodiment the load buffer comprises 94 mM sodium chloride. In one embodiment the load buffer comprises 96 mM sodium chloride. In one embodiment the load buffer comprises 98 mM sodium chloride. In one embodiment the load buffer comprises 105 mM sodium chloride.

In one embodiment of the invention, the load buffer comprises acetate. In one embodiment the load buffer comprises acetate, pH 4.9-5.1. In one embodiment the load buffer comprises acetate at pH, 4.9, 5.0, or 5.1. In one embodiment the load buffer comprises acetate at pH, 4.9, 4.95, 5.0, 5.05, or 5.1. In one embodiment the load buffer comprises acetate, pH of 5.0±0.05 to 5.0±0.1. In one embodiment the load buffer comprises acetate at pH, 5.0. In one embodiment the load buffer comprises 100 mM acetate. In one embodiment the load buffer comprises 100 mM acetate, pH 4.9-5.1. In one embodiment the load buffer comprises 100 mM acetate at pH, 4.9, 5.0, or 5.1. In one embodiment the load buffer comprises 100 mM acetate at pH, 4.9, 4.95, 5.0, 5.05, or 5.1. In one embodiment the load buffer comprises 100 mM acetate, pH 5.0±0.05% to pH 5.0±0.1%.

In one embodiment the load buffer comprises acetate and 94 mM-105 mM sodium chloride. In one embodiment the load buffer comprises acetate and 94 mM-96 mM sodium chloride. In one embodiment the load buffer comprises acetate and 96 mM-105 mM sodium chloride. In a related embodiment the load buffer comprises acetate and 94 mM sodium chloride. In a related embodiment the load buffer comprises acetate and 96 mM sodium chloride. In a related embodiment the load buffer comprises acetate and 98 mM sodium chloride. In a related embodiment the load buffer comprises acetate and 105 mM sodium chloride. In a related embodiment, the acetate concentration is 100 mM.

In a related embodiment the load buffer comprises acetate, 94 mM-105 mM sodium chloride, pH 4.9-5.1. In a related embodiment the load buffer comprises acetate, 94 mM-105 mM sodium chloride, pH of 4.9, 5.0 or 5.1. In a related embodiment the load buffer comprises acetate, 94-105 mM sodium chloride, pH of 4.9, 4.95, 5.0, 5.05, or 5.1. In a related embodiment the load buffer comprises acetate, 94 mM-105 mM sodium chloride, pH of 5.0. In a related embodiment, the acetate concentration is 100 mM.

In one embodiment the load buffer comprises 100 mM acetate, 94 mM-105 mM sodium chloride, pH 5.0±0.05 to 5.0±0.1. In a related embodiment the load buffer comprises 100 mM acetate, 94 mM-105 mM sodium chloride, pH 4.9-5.1. In a related embodiment the load buffer comprises 100 mM acetate, 94 mM-105 mM sodium chloride, pH 4.9, 4.95, 5.0, 5.05, or 5.1. In a related embodiment the load buffer comprises 100 mM acetate, 94 mM-105 mM sodium chloride, pH 4.9, 5.0, or 5.1. In a related embodiment the load buffer comprises 100 mM acetate, 94 mM-105 mM sodium chloride, pH 5.0.

In one embodiment at least one wash buffer comprises 94-96 mM sodium chloride. In one embodiment at least one wash buffer comprises 96-105 mM sodium chloride. In one embodiment at least one wash buffer comprises 94, 95, 96, 97, 98, 99, 100, 101, 102, 103, 104, or 105 mM sodium chloride. In one embodiment at least one wash buffer comprises 94 mM sodium chloride. In one embodiment at least one wash buffer comprises 96 mM sodium chloride. In one embodiment at least one wash buffer comprises 98 mM sodium chloride. In one embodiment at least one wash buffer comprises 105 mM sodium chloride.

In one embodiment of the invention, at least one wash buffer comprises acetate. In one embodiment at least one wash buffer comprises acetate, pH 4.9-5.1. In one embodiment at least one wash buffer comprises acetate at pH, 4.9, 5.0, or 5.1. In one embodiment the wash buffer comprises acetate at pH, 4.9, 4.95, 5.0, 5.05, or 5.1. In one embodiment at least one wash buffer comprises acetate, pH of 5.0±0.05 to 5.0±0.1. In one embodiment at least one wash buffer comprises 100 mM acetate. In one embodiment at least one wash buffer comprises 100 mM acetate, pH 4.9-5.1. In one embodiment at least one wash buffer comprises 100 mM acetate at pH, 4.9, 5.0, or 5.1. In one embodiment the wash buffer comprises 100 mM acetate at pH, 4.9, 4.95, 5.0, 5.05, or 5.1. In one embodiment at least one wash buffer comprises 100 mM acetate, pH 5.0±0.05% to pH 5.0±0.1%.

In one embodiment at least one wash buffer comprises acetate, 94 mM-105 mM sodium chloride. In one embodiment at least one wash buffer comprises acetate, 94 mM-96 mM sodium chloride. In one embodiment at least one wash buffer comprises acetate, 96 mM-105 mM sodium chloride. In a related embodiment at least one wash buffer comprises acetate, 94 mM sodium chloride. In a related embodiment at least one wash buffer comprises acetate, 96 mM sodium chloride. In a related embodiment at least one wash buffer comprises acetate, 98 mM sodium chloride. In a related embodiment at least one wash buffer comprises acetate, 105 mM sodium chloride. In a related embodiment, the acetate concentration is 100 mM.

In one embodiment at least one wash buffer comprises acetate, 94 mM-105 mM sodium chloride, pH 5.0±0.05 to 5.0±0.1. In a related embodiment at least one wash buffer comprises acetate, 94 mM-105 mM sodium chloride, pH 4.9-5.1. In a related embodiment at least one wash buffer comprises acetate, 94 mM-105 mM sodium chloride, pH of 4.9, 5.0 or 5.1. In a related embodiment at least one wash buffer comprises acetate, 94 mM-105 mM sodium chloride, pH 4.9, 4.95, 5.0, 5.05, or 5.1. In a related embodiment at least one wash buffer comprises acetate, 94 mM-105 mM sodium chloride, pH of 5.0. In a related embodiment, the acetate concentration is 100 mM.

In one embodiment at least one wash buffer comprises 100 mM acetate, 94 mM-105 mM sodium chloride, pH 5.0±0.05 to 5.0±0.1. In one embodiment at least one wash buffer comprises 100 mM acetate, 94 mM-105 mM sodium chloride, pH 4.9-5.1. In a related embodiment at least one wash buffer comprises 100 mM acetate, 94 mM-105 mM sodium chloride, pH 4.9, 5.0, or 5.1. In a related embodiment at least one wash buffer comprises 100 mM acetate, 94 mM-105 mM sodium chloride, pH 4.9, 4.95, 5.0, 5.05, or 5.1. In a related embodiment at least one wash buffer comprises 100 mM acetate, 94 mM-105 mM sodium chloride, pH 5.0.

In one embodiment there is at least one additional wash step with a different wash buffer. In one embodiment at least one additional wash is a second wash. In one embodiment at least one additional wash buffer comprises 0-26 mM sodium chloride. In one embodiment at least one additional wash buffer comprises 0, 23, 24, 25, or 26 mM sodium chloride. In one embodiment at least one additional wash buffer comprises 0 mM sodium chloride. In one embodiment at least one additional wash buffer comprises 23 mM sodium chloride. In one embodiment at least one additional wash buffer comprises 24 mM sodium chloride. In one embodiment at least one additional wash buffer comprises 25 mM sodium chloride. In one embodiment at least one additional wash buffer comprises 26 mM sodium chloride.

In one embodiment of the invention, at least one additional wash buffer comprises acetate. In one embodiment at least one additional wash buffer comprises acetate, pH 4.9-5.1. In one embodiment at least one additional wash buffer comprises acetate at pH, 4.9, 5.0, or 5.1. In one embodiment at least one additional wash buffer comprises acetate at pH, 4.9, 4.95, 5.0, 5.05, or 5.1. In one embodiment at least one additional wash buffer comprises acetate, pH of 5.0±0.05 to 5.0±0.1. In one embodiment at least one additional wash buffer comprises 100 mM acetate. In one embodiment at least one additional wash buffer comprises 100 mM acetate, pH 4.9-5.1. In one embodiment at least one additional wash buffer comprises 100 mM acetate at pH, 4.9, 5.0, or 5.1. In one embodiment at least one additional wash buffer comprises 100 mM acetate at pH, 4.9, 4.95, 5.0, 5.05, or 5.1. In one embodiment at least one additional wash buffer comprises 100 mM acetate, pH 5.0±0.05% to pH 5.0±0.1%.

In one embodiment at least one wash buffer comprises acetate and sodium chloride followed by at least one additional wash. In one embodiment at least one wash buffer comprises acetate, 94-105 mM sodium chloride, followed by at least one additional wash. In one embodiment at least one wash buffer comprises acetate, 94-105 mM sodium chloride, followed by at least one additional wash buffer comprising 0-26 mM sodium chloride. In a related embodiment at least one additional wash buffer comprises 23-26 mM sodium chloride. In a related embodiment at least one additional wash buffer comprises 0 mM sodium chloride. In a related embodiment at least one additional wash buffer comprises 23 mM sodium chloride. In a related embodiment at least one additional wash buffer comprises 24 mM sodium chloride. In a related embodiment at least one additional wash buffer comprises 25 mM sodium chloride. In a related embodiment at least one additional wash buffer comprises 26 mM sodium chloride.

In one embodiment at least one wash buffer comprises acetate and sodium chloride followed by at least one additional wash. In one embodiment at least one wash buffer comprises acetate, 94-105 mM sodium chloride, followed by at least one additional wash comprising acetate. In one embodiment at least one wash buffer comprises acetate, 94-105 mM sodium chloride, followed by at least one additional wash buffer comprising acetate, 0-26 mM sodium chloride. In a related embodiment at least one additional wash buffer comprises acetate, 23-26 mM sodium chloride. In a related embodiment at least one additional wash buffer comprises acetate, 0 mM sodium chloride. In a related embodiment at least one additional wash buffer comprises acetate, 23 mM sodium chloride. In a related embodiment at least one additional wash buffer comprises acetate, 24 mM sodium chloride. In a related embodiment at least one additional wash buffer comprises acetate, 25 mM sodium chloride. In a related embodiment at least one additional wash buffer comprises acetate, 26 mM sodium chloride. In one embodiment at least one wash buffer comprises acetate, 94 mM sodium chloride, followed by an additional wash buffer comprising acetate, 23-24 mM sodium chloride. In one embodiment at least one wash buffer comprises acetate, 96 mM sodium chloride, followed by an additional wash buffer comprising acetate, 25 mM sodium chloride. In one embodiment at least one wash buffer comprises acetate, 98 mM sodium chloride, followed by an additional wash buffer comprising acetate, 26 mM sodium chloride. In one embodiment at least one wash buffer comprises acetate, 105 mM sodium chloride, followed by an additional wash buffer comprising acetate, 0 mM sodium chloride. In one embodiment, the acetate concentration is 100 mM.

In one embodiment the additional wash is a second wash. In a related embodiment the second wash buffer comprises 0-26 mM sodium chloride. In one embodiment least one wash buffer comprises acetate, 94-105 mM sodium chloride, followed by a second wash buffer comprising acetate, 0-26 mM sodium chloride. In one embodiment, the acetate concentration is 100 mM.

In one embodiment at least one wash buffer comprises acetate, 94-105 mM sodium chloride, followed by at least one additional wash buffer comprising acetate, 0-26 mM sodium chloride where the pH of the buffers is the same or different. In one embodiment, the pH of one or more of the wash buffers is pH 4.9-5.1. In one embodiment, the pH of one or more of the wash buffers is pH 4.9, 4.95, 5.0, 5.05, or 5.1. In one embodiment, the pH of one or more of the wash buffers is pH 4.9, 5.0, or 5.1. In one embodiment, the pH of one or more of the wash buffers is pH 5.0. In one embodiment, the pH of one or more of the wash buffers is pH 5.0±0.05% to pH 5.0±0.1%. In one embodiment the acetate concentration of one or more of the wash buffers is 100 mM.

In one embodiment the equilibration buffer comprises 94-96 mM sodium chloride. In one embodiment the equilibration buffer comprises 96-105 mM sodium chloride. In one embodiment the equilibration buffer comprises 94, 95, 96, 97, 98, 99, 100, 101, 102, 103, 104, or 105 mM sodium chloride. In one embodiment the equilibration buffer comprises 94 mM sodium chloride. In one embodiment the equilibration buffer comprises 96 mM sodium chloride. In one embodiment the equilibration buffer comprises 105 mM sodium chloride.

In one embodiment of the invention, the equilibration buffer comprises acetate. In one embodiment the equilibration buffer comprises acetate, pH 4.9-5.1. In one embodiment the equilibration buffer comprises acetate at pH 4.9, 5.0, or 5.1. In one embodiment the equilibration buffer comprises acetate at pH 5.0. In one embodiment the equilibration buffer comprises acetate, pH of 5.0±0.05 to 5.0±0.1. In one embodiment the equilibration buffer comprises 100 mM acetate. In one embodiment the equilibration buffer comprises 100 mM acetate, pH 4.9-5.1. In one embodiment the equilibration buffer comprises 100 mM acetate at pH, 4.9, 4.95, 5.0, 5.05, or 5.1. In one embodiment the equilibration buffer comprises 100 mM acetate at pH, 4.9, 5.0, or 5.1. In one embodiment the equilibration buffer comprises 100 mM acetate, pH 5.0±0.05% to pH 5.0±0.1%.

In one embodiment the equilibration buffer comprises acetate, 94 mM-105 mM sodium chloride. In one embodiment the equilibration buffer comprises acetate, 94 mM-96 mM sodium chloride. In one embodiment the equilibration buffer comprises acetate, 96 mM-105 mM sodium chloride. In a related embodiment the equilibration buffer comprises acetate, 94 mM sodium chloride. In a related embodiment the equilibration buffer comprises acetate, 96 mM sodium chloride. In a related embodiment the equilibration buffer comprises acetate, 105 mM sodium chloride. In one embodiment, the acetate concentration is 100 mM.

In one embodiment the equilibration buffer comprises acetate, 94 mM-105 mM sodium chloride, pH 5.0±0.05 to 5.0±0.1. In a related embodiment the equilibration buffer comprises acetate, 94 mM-105 mM sodium chloride, pH 4.9-5.1. In a related embodiment the equilibration buffer comprises acetate, 94 mM-105 mM sodium chloride, pH of 4.9, 4.95, 5.0, 5.05, or 5.1. In a related embodiment the equilibration buffer comprises acetate, 94 mM-105 mM sodium chloride, pH of 4.9, 5.0 or 5.1. In a related embodiment the equilibration buffer comprises acetate, 94 mM-105 mM sodium chloride, pH of 5.0. In one embodiment, the acetate concentration is 100 mM.

In one embodiment the equilibration buffer comprises 100 mM acetate, 94 mM-105 mM sodium chloride, pH 5.0±0.05 to 5.0±0.1. In a related embodiment the equilibration buffer comprises 100 mM acetate, 94 mM-105 mM sodium chloride, pH 4.9-5.1. In a related embodiment the equilibration buffer comprises 100 mM acetate, 94 mM-105 mM sodium chloride, pH of 4.9, 4.95, 5.0, 5.01, or 5.1. In a related embodiment the equilibration buffer comprises 100 mM acetate, 94 mM-105 mM sodium chloride, pH of 4.9, 5.0 or 5.1. In a related embodiment the equilibration buffer comprises 100 mM acetate, 94 mM-105 mM sodium chloride, pH of 5.0.

In one embodiment the composition is loaded at 10-27 g/L. In a related embodiment the composition is loaded at 10-25 g/L. In a related embodiment the composition is loaded at 10-23 g/L. In a related embodiment the composition is loaded at 10-15 g/L. In a related embodiment the composition is loaded at 15-27 g/L. In a related embodiment the composition is loaded at 15-25 g/L. In a related embodiment the composition is loaded at 15-23 g/L. In a related embodiment the composition is loaded at 23-27 g/L. In a related embodiment the composition is loaded at 23-25 g/L. In a related embodiment the composition is loaded at 25-27 g/L. In one embodiment the composition is loaded at 10, 15, 23, 25 or 27 g/L. In one embodiment the composition is loaded at 10 g/L. In one embodiment the composition is loaded at 15 g/L. In one embodiment the composition is loaded at 23 g/L. In one embodiment the composition is loaded at 25 g/L. In one embodiment the composition is loaded at 27 g/L.

In one embodiment the multispecific protein is eluted from the cation exchange resin by a gradient. In a related embodiment the gradient is linear. In a related embodiment the gradient is a salt gradient.

In a related embodiment the low pI impurity is a product-related impurity. In a related embodiment at least one product-related impurity is a half antibody or 2×, 3×, or 4× light chain-mis-assembly.

In one embodiment the multispecific protein is a bispecific protein. In one embodiment the multispecific protein is a bispecific antibody.

In one embodiment the cation exchange chromatography medium is a resin. In one embodiment the second chromatography medium is a resin. In a related embodiment the second chromatography resin is selected from an anion exchange chromatography resin, cation exchange chromatography resin, multi-modal chromatography resin, hydrophobic interaction chromatography resin, and hydroxyapatite chromatography resin.

The invention provides a purified, multispecific protein produced according to the methods described herein. The invention provides an isolated, purified, recombinant multispecific proteins made according to the methods described herein.

The invention provides a pharmaceutical composition comprising the isolated, purified, recombinant multispecific protein of interest according to the methods described herein.

“Multispecific”, “multispecific protein”, and “multispecific antibody” are used interchangeably herein to refer to proteins that are recombinantly engineered to simultaneously bind and neutralize at least two different antigens or at least two different epitopes on the same antigen. For example, multispecific proteins can be engineered to target immune effectors and cytotoxic agents to tumors or infectious agents. These multispecific proteins have been found useful for a variety of applications such as in cancer immunotherapy by redirecting immune effector cells to tumor cells, modifying cell signaling by blocking signaling pathways, targeting tumor angiogenesis, blocking cytokines, and as pre-targeted delivery vehicles for drugs, such as delivery of chemotherapeutic agents, radiolabels (to improve detection sensitivity) and nanoparticles (directed to specific cells/tissues, such as cancer cells).

The most common and most diverse of the multispecific proteins are those that bind two antigens, referred to interchangeably herein as “bispecific”, “bispecific proteins”, and “bispecific antibody”. Bispecific proteins can be grouped in two broad categories: immunoglobulin G (IgG)-like molecules and non-IgG-like molecules. IgG-like molecules retain Fc-mediated effector functions, such as antibody-dependent cell mediated cytotoxicity (ADCC), complement-dependent cytotoxicity (CDC), and antibody-dependent cellular phagocytosis (ADCP), the Pc region helps improve solubility and stability and facilitate some purification operations. Non-IgG-like molecules are smaller, enhancing tissue penetration. (Sedykh et al., Drug Design, Development and Therapy 18(12), 195-208, 2018; Fan et al., J Hematol & Oncology 8:130-143, 2015; Spiess et al., Mol Immunol 67, 95-106, 2015); Williams et al., Chapter 41 Process Design for Bispecific Antibodies in Biopharmaceutical Processing Development, Design and Implementation of Manufacturing Processes, Jagschies et al., eds., 2018, pages 837-855. Bispecific proteins are sometimes used as a framework for additional components having binding specificities to different antigens or numbers of epitopes, increasing the binding specificity of the molecule.

The formats for bispecific proteins, which include bispecific antibodies, are constantly evolving and include, but are not limited to, quadromas, knobs-in-holes, cross-Mabs, dual variable domains IgG (DVD-IgG), IgG-single chain Fv (scFv), scFv-CH3 KIH, dual action Fab (DAF), half-molecule exchange, κλ-bodies, tandem scFv, scFv-Fc, diabodies, single chain diabodies (scDiabodies), scDiabodies-CH3, triple body, miniantibody, minibody, TriBi minibody, tandem diabodies, scDiabody-HAS, Tandem scFv-toxin, dual-affinity retargeting molecules (DARTs), nanobody, nanobody-HSA, dock and lock (DNL), strand exchange engineered domain SEEDbody, Triomab, leucine zipper (LUZ-Y), XmAb®; Fab-arm exchange, DutaMab, DT-IgG, charged pair, Fcab, orthogonal Fab, IgG(H)-scFv, scFV-(H)IgG, IgG(L)-scFV, IgG(L1H1)-Fv, IgG(H)-V, V(H)-IgG, IgG(L)-V V(L)-IgG, KIH IgG-scFab, 2scFV-IgG, IgG-2scFv, scFv4-Ig, Zybody, DVI-Ig4 (four-in-one), Fab-scFv, scFv-CH-CL-scFV, F(ab′)2-scFv2, scFv-KIH, Fab-scFv-Fc, tetravalent HCAb, scDiabody-Fc, diabody-Fc, intrabody, ImmTAC, HSABody, IgG-IgG, Cov-X-Body, scFv1-PEG-scFv2, single chain bispecific antibody constructs, single chain bispecific T cell engagers (BITE), bi-specific T cell engagers, and half-life extended bispecific T cell engagers (HLE BITE) (Fan supra; Spiess supra; Sedykh supra; Seimetz et al., Cancer Treat Rev 36(6) 458-67, 2010; Shulka and Norman, Chapter 26 Downstream Processing of Fc Fusion Proteins, Bispecific Antibodies, and Antibody-Drug Conjugates, in Process Scale Purification of Antibodies Second Edition, Uwe Gottswchalk editor, p 559-594, John Wiley & Sons, 2017; Moore et al., MAbs 3:6, 546-557, 2011

In some embodiments, bispecific proteins may include blinatumomab, catumaxomab, ertumaxomab, solitomab, targomiRs, lutikizumab (ABT981), vanucizumab (RG7221), remtolumab (ABT122), ozoralixumab (ATN103), floteuzmab (MGD006), pasotuxizumab (AMG112, MT112), lymphomun (FBTA05), (ATN-103), AMG211 (MT111, Medi-1565), AMG330, AMG420 (B1836909), AMG-110 (MT110), MDX-447, TF2, rM28, HER2Bi-aATC, GD2Bi-aATC, MGD006, MGD007, MGD009, MGD010, MGD011 (JNJ64052781), IMCgp100, indium-labeled IMP-205, xm734, LY3164530, OMP-305BB3, REGN1979, COV322, ABT112, ABT165, RG-6013 (ACE910), RG7597 (MEDH7945A), RG7802, RG7813(RO6895882), RG7386, BITS7201A (RG7990), RG7716, BFKF8488A (RG7992), MCLA-128, MM-111, MM141, MOR209/ES414, MSB0010841, ALX-0061, ALX0761, ALX0141; BII034020, AFM13, AFM11, SAR156597, FBTA05, PF06671008, GSK2434735, MEDI3902, MEDI0700, MEDI7352, as well as the molecules or variants or analogs thereof and biosimilars of any of the foregoing.

Multispecific proteins also include trispecific antibodies, tetravalent bispecific antibodies, multispecific proteins without antibody components such as dia-, tria- or tetrabodies, minibodies, and single chain proteins capable of binding multiple targets. Coloma, M. J., et. al., Nature Biotech. 15 (1997) 159-163.

In some embodiments, multispecific proteins of interest bind, neutralize and/or interact specifically to one or more CD proteins, HER receptor family proteins, cell adhesion molecules, growth factors, nerve growth factors, fibroblast growth factors, transforming growth factors (TGF), insulin-like growth factors, osteoinductive factors, insulin and insulin-related proteins, coagulation and coagulation-related proteins, colony stimulating factors (CSFs), other blood and serum proteins blood group antigens; receptors, receptor-associated proteins, growth hormones, growth hormone receptors, T-cell receptors; neurotrophic factors, neurotrophins, relaxins, interferons, interleukins, viral antigens, lipoproteins, integrins, rheumatoid factors, immunotoxins, surface membrane proteins, transport proteins, homing receptors, addressins, regulatory proteins, and immunoadhesins.

In some embodiments multispecific proteins of interest bind, neutralize and/or interact with one or more of the following, alone or in any combination: CD proteins including but not limited to CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, for example, vascular endothelial growth factor (“VEGF”); VEGFR2, growth hormone, thyroid stimulating hormone, follicle stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors, including, for instance, aFGF and bFGF, epidermal growth factor (EGF), Cripto, transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors, insulins and insulin-related proteins, including but not limited to insulin, insulin A-chain, insulin B-chain, proinsulin, and insulin-like growth factor binding proteins; (coagulation and coagulation-related proteins, such as, among others, factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs), including the following, among others, M-CSF, GM-CSF, and G-CSF, other blood and serum proteins, including but not limited to albumin, IgE, and blood group antigens, receptors and receptor-associated proteins, including, for example, flk2/flt3 receptor, obesity (OB) receptor, growth hormone receptors, and T-cell receptors; neurotrophic factors, including but not limited to, bone-derived neurotrophic factor (BDNF) and neurotrophin-3, -4, -5, or -6 (NT-3, NT-4, NT-5, or NT-6); relaxin A-chain, relaxin B-chain, and prorelaxin, interferons, including for example, interferon-alpha, -beta, and -gamma, interleukins (ILs), e.g., IL-1 to IL-10, IL-12, IL-15, IL-17, IL-23, IL-12/IL-23, IL-2Ra, IL1-R1, IL-6 receptor, IL-4 receptor and/or IL-13 to the receptor, IL-13RA2, or IL-17 receptor, IL-IRAP, viral antigens, including but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, Dnase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, addressins, regulatory proteins, immunoadhesins, antigen-binding proteins, somatropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, glanglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, NKG2D-1, programmed cell death protein 1 and ligand, PD1 and PDL1, mannose receptor/hCGβ, hepatitis-C virus, mesothelin dsFv[PE38 conjugate, Legionella pneumophila (IIy), IFN gamma, interferon gamma induced protein 10 (IP10), IFNAR, TALL-1, TNFα, TL1A, thymic stromal lymphopoietin (TSLP), proprotein convertase subtilisin/Kexin Type 9 (PCSK9), stem cell factors, Flt-3, calcitonin gene-related peptide (CGRP), OX40L, α4β7, platelet specific (platelet glycoprotein Iib/IIIb (PAC-1), transforming growth factor beta (TFGβ), STEAP1, Zona pellucida sperm-binding protein 3 (ZP-3), TWEAK, platelet derived growth factor receptor alpha (PDGFRα), sclerostin, and biologically active fragments or variants of any of the foregoing.

In some embodiments, multispecific proteins of interest may include bispecific antibodies that specifically bind to combinations including CD3 and CD19, EpCAM, CEA, PSA, CD33, BCMA, Her2, CD20, P-cadherin, CD123, gpA33, or B7H3. In some embodiments, bispecific antibodies of interest may include bispecific antibodies that specifically bind to combinations including IL1α+IL1β.

The multispecific proteins can be of scientific or commercial interest, particularly bispecific-based therapeutics. Multispecific proteins can be produced in various ways, most commonly by recombinant animal cell lines using cell culture methods. The multispecific proteins may be produced intracellularly or secreted into the culture medium from which it can be recovered and/or collected and may be referred to interchangeably as “recombinant multispecific protein”, “recombinant multispecific antibody. The term “isolated multispecific protein”, “isolated recombinant multispecific antibody” refer to a multispecific protein that that have been purified away from proteins, polypeptides, DNA, and/or other contaminants or impurities that would interfere with its therapeutic, diagnostic, prophylactic, research, or other use. Also included are “recombinant bispecific protein”, “recombinant bispecific antibody”, “isolated recombinant bispecific protein”, and “isolated recombinant bispecific antibody”. Multispecific proteins of interest include multispecific antibodies that exert a therapeutic effect by binding two or more targets, particularly targets among those listed below, including targets derived therefrom, targets related thereto, and modifications thereof.

The invention provides a method of purifying a multispecific protein from a composition comprising the multispecific protein and at least one product-related impurity, the method comprising equilibrating a cation exchange chromatography medium with an equilibration buffer comprising 94-105 mM Sodium chloride; loading the composition on to the cation exchange medium in a load buffer comprises 94-105 mM Sodium chloride; washing the column with at least one wash buffer comprising 94-105 mM Sodium chloride; and eluting the multispecific protein from the cation exchange chromatography medium.

By “purifying” is meant increasing the degree of purity of the multispecific protein in the composition by removing (partially or completely) at least one product-related impurity from the composition. Recovery and purification of multispecific proteins is accomplished by the downstream unit operations, in particular, those operations involving ion exchange chromatography, resulting in a more “homogeneous” multispecific protein composition that meets yield and product quality targets (such as reduced product-related impurities, increased product quality and the like).

“Product-related impurity” refers to product-related variants of the multispecific protein of interest. In some instances, these impurities have a pI that is lower than the main product in an elution peak. Product-related impurities include, for example, homodimers, half antibodies, aggregates, antibody fragments and various combinations of antibody fragments, and light chain mis-assemblies, such as 2×LC, 3×LC, or 4×LC, high molecular weight (HMW) species, low molecular weight (LMW) species. “Half antibodies” refer to a product-related impurity that can form, for example, due to incomplete assembly or disruption of the interaction between the two heavy chain polypeptides. Half antibodies comprise a single light chain polypeptide and a single heavy chain polypeptide. “Homodimers” refer to a product-related impurity that can, for example, form when heavy and light chains having specificity for the same target recombine with each other instead of pairing with heavy and light chains that have specificity to a different target to form a desired bispecific heterodimer. This typically occurs during expression in the host cell. For multispecific constructs that require multiple chains (such as light chains, LCs) to pair correctly via engineered residues (such as charged paired mutations, knob-hole, etc), it is possible to still have impurities where there is a mismatch between LC and HC, wherein LC1 instead of pairing with HC1, incorrectly pairs with HC2 (2×LC1), and vice versa (2×LC2). If the multispecific protein is bivalent, having two sites for binding to each antigen of interest, it is possible to have 3× LC1, 4× LC1, and other combinations of mispaired species.

The invention provides a method of reducing low pI impurities in the eluate from cation exchange chromatography, the method comprises equilibrating a cation exchange chromatography medium with an equilibration buffer comprising 94-105 mM Sodium chloride; loading the composition on to the cation exchange medium in a load buffer comprises 94-105 mM Sodium chloride; washing the column with at least one wash buffer comprising 94-105 mM Sodium chloride; and eluting the multispecific protein from the cation exchange chromatography medium; wherein the cation exchange chromatography eluate has reduced low pI impurities compared to the cation exchange chromatography eluate recovered in a corresponding method in which no sodium chloride is used in the equilibration, load, and wash steps.

As disclosed herein, the pI of product-related impurities may be similar to the pI of the desired multispecific protein. These product-related impurities are found in the eluate peak with the main product. They have a slightly lower pI so they elute just prior to the main product, as pre-peaks. The “isoelectric point” or “pI” of a protein, refers to the pH at which the positive charge balances the negative charge of the protein. The pI can be calculated/determined using known methods such as from the net charge of the amino acid residues of the protein or by isoelectric focusing. Product-related impurities having a lower pI than the main product are more acidic than the main product.

The invention provides method of producing an isolated, purified, recombinant multispecific protein of interest, the method comprising establishing a cell culture in a bioreactor with a host cell expressing the multispecific protein; culturing the host cells to express the multispecific protein; harvesting the recombinant multispecific protein; affinity purifying the harvested recombinant multispecific protein; inactivating virus at low pH in the eluate pool from the affinity purification and neutralizing the pool; equilibrating a cation exchange chromatography medium with an equilibration buffer comprising 94-105 mM Sodium chloride; loading the neutralized affinity purified recombinant multispecific protein on to the equilibrated cation exchange medium in a load buffer comprises 94-105 mM Sodium chloride; washing the cation exchange medium with a wash buffer comprising 94-105 mM Sodium chloride, followed by a second wash buffer comprising 0-26 mM Sodium chloride; eluting the multispecific protein from the cation exchange chromatography medium; and loading the cation exchange chromatography eluate comprising the recombinant multispecific protein onto a second chromatography resin in flow through mode; and concentrating the purified recombinant multispecific protein in a formulation buffer.

Expression systems and constructs in the form of plasmids, expression vectors, transcription or expression cassettes that comprise at least one nucleic acid encoding a multispecific protein are provided herein, as well host cells comprising such expression systems or constructs. As used herein, “vector” means any molecule or entity (e.g., nucleic acid, plasmid, bacteriophage, transposon, cosmid, chromosome, virus, virus capsid, virion, naked DNA, complexed DNA and the like) suitable for use to transfer and/or transport multispecific protein encoding information into a host cell and/or to a specific location and/or compartment within a host cell. Vectors can include viral and non-viral vectors, non-episomal mammalian vectors. Vectors are often referred to as expression vectors, for example, recombinant expression vectors and cloning vectors. The vector may be introduced into a host cell to allow replication of the vector itself and thereby amplify the copies of the polynucleotide contained therein. The cloning vectors may contain sequence components that generally include, without limitation, an origin of replication, promoter sequences, transcription initiation sequences, enhancer sequences, and selectable markers. These elements may be selected as appropriate by a person of ordinary skill in the art.

“Cell” or “Cells” include any prokaryotic or eukaryotic cell. Cells can be either ex vivo, in vitro or in vivo, either separate or as part of a higher structure such as a tissue or organ. Cells include “host cells”, also referred to as “cell lines”, which are genetically engineered to express a multispecific protein of commercial or scientific interest. Host cells are typically derived from a lineage arising from a primary culture that can be maintained in culture for an unlimited time. Genetically engineering the host cell involves transfecting, transforming or transducing the cells with a recombinant polynucleotide molecule, and/or otherwise altering (e.g., by homologous recombination and gene activation or fusion of a recombinant cell with a non-recombinant cell) to cause the host cell to express a desired recombinant multispecific protein. Methods and vectors for genetically engineering cells and/or cell lines to express multispecific proteins of interest are well known to those of skill in the art.

A host cell can be any prokaryotic cell (for example, Escherichia coli (E. coli)) or eukaryotic cell (for example, yeast, insect, or animal cells, in particular mammalian cells (e.g., CHO cells)). Vector DNA can be introduced into prokaryotic or eukaryotic cells via conventional transformation or transfection techniques.

In one embodiment, the cell is a host cell. Host cells, when cultured under appropriate conditions, express the multispecific protein of interest that can be subsequently collected from the culture medium (if the host cell secretes it into the medium) or directly from the host cell producing it (if it is not secreted). The selection of an appropriate host cell will depend upon various factors, such as desired expression levels, protein modifications that are desirable or necessary for activity (such as glycosylation or phosphorylation) and ease of folding into a biologically active molecule.

By “culture” or “culturing” is meant the growth and propagation of cells outside of a multicellular organism or tissue. Suitable culture conditions for mammalian cells are known in the art. Cell culture media and tissue culture media are used interchangeably to refer to media suitable for growth of a host cell during in vitro cell culture. Typically, cell culture media contains a buffer, salts, energy source, amino acids, vitamins and trace essential elements. Any media capable of supporting growth of the appropriate host cell in culture can be used. Cell culture media, which may be further supplemented with other components to maximize cell growth, cell viability, and/or recombinant protein production in a particular cultured host cell, are commercially available and include RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series, among others, which can be obtained from the American Type Culture Collection or SAFC Biosciences, as well as other vendors. Cell culture media can be serum-free, protein-free, growth factor-free, and/or peptone-free media. Cell culture may also be enriched by the addition of nutrients and used at greater than its usual, recommended concentrations.

Various media formulations can be used during the life of the culture, for example, to facilitate the transition from one stage (e.g., the growth stage or phase) to another (e.g., the production stage or phase) and/or to optimize conditions during cell culture (e.g. concentrated media provided during perfusion culture). A growth medium formulation can be used to promote cell growth and minimize protein expression. A production medium formulation can be used to promote production of the protein of interest and maintenance of the cells, with a minimal of new cell growth). A feed media, typically a media containing more concentrated components such as nutrients and amino acids, which are consumed during the course of the production phase of the cell culture may be used to supplement and maintain an active culture, particularly a culture operated in fed batch, semi-perfusion, or perfusion mode. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount.

A growth phase may occur at a higher temperature than a production phase. For example, a growth phase may occur at a first temperature from about 35° C. to about 38° C., and a production phase may occur at a second temperature from about 29° C. to about 37° C., optionally from about 30° C. to about 36° C. or from about 30° C. to about 34° C. In addition, chemical inducers of protein production, such as, for example, caffeine, butyrate, and hexamethylene bisacetamide (HMBA), may be added at the same time as, before, and/or after a temperature shift. If inducers are added after a temperature shift, they can be added from one hour to five days after the temperature shift, optionally from one to two days after the temperature shift. pH may also be shifted during culture, either independently or in combination with other methods.

Host cells may be cultured in suspension or in an adherent form, attached to a solid substrate. Cell cultures can be established in fluidized bed bioreactors, hollow fiber bioreactors, roller bottles, shake flasks, or stirred tank bioreactors, with or without microcarriers

Cell cultures can be operated in a batch, fed batch, continuous, semi-continuous, or perfusion mode Mammalian cells, such as CHO cells, may be cultured in bioreactors at a smaller scale of less than 100 ml to less than 1000 mls. Alternatively, larger scale bioreactors that contain 1000 mls to over 20,000 liters of media can be used. Large scale cell cultures, such as for clinical and/or commercial scale biomanufacturing of protein therapeutics, may be maintained for weeks and even months, while the cells produce the desired protein(s).

Since product-related impurities, such as homodimers, half antibodies, 2× LC-mis alignments and the like can resemble the desired multispecific protein, strategies and techniques such as knob and hole, CrossMab, DVD IgG, and others have been developed to increase the selectivity for the desired multispecific protein during cell culture. However, there will still be some amount of product-related impurities that are produced which must be removed during downstream processing.

The resulting expressed recombinant multispecific protein can then be harvested from the cell culture media. Methods for harvesting proteins from suspension cells are known in the art and include, but are not limited to, acid precipitation, accelerated sedimentation such as flocculation, separation using gravity, centrifugation, acoustic wave separation, filtration, including membrane filtration, using ultrafilters, microfilters, tangential flow filters, alternating tangential flow, depth, and alluvial filtration filters. Recombinant proteins expressed by prokaryotes are retrieved from inclusion bodies in the cytoplasm by redox folding processes known in the art.

The harvested multispecific protein can then be purified, or partially purified, away from any impurities, such as remaining cell culture media, cell extracts, undesired components, host cell proteins, improperly expressed proteins, product-related impurities, and the like, through one or more downstream purification operations.

Purification of the multispecific protein from the harvested cell culture fluid can begin with capture chromatography that makes use of resins and/or membranes containing agents that will bind to the recombinant multispecific protein of interest, for example affinity chromatography, size exclusion chromatography, ion exchange chromatography, hydrophobic interaction chromatography (HIC), immobilized metal affinity chromatography (IMAC), and the like. Such materials are known in the art and are commercially available. Affinity chromatography options may comprise a substrate-binding capture mechanism, an aptamer-binding capture mechanism, and a cofactor-binding capture mechanism, for example. For multispecific proteins containing an Fc component, an antibody- or antibody fragment-binding capture mechanism such as Protein A, Protein G, Protein A/G, Protein L can be used.

At any point in the downstream process virus inactivation and/or virus filtration can be performed to remove viral matter from the purified multispecific protein solution. One method for achieving virus inactivation is incubation at low pH or other solution conditions for achieving the inactivation of viruses. Low pH virus inactivation can be followed with a neutralization unit operation that readjusts the viral inactivated solution to a pH more compatible with the requirements of the following unit operations. Typically, neutralization is at pH 5-7. Viral inactivated or neutralized viral inactivated pools may also be followed by filtration, such as depth filtration, to remove any resulting turbidity or precipitation. Sterile filtration is typically performed along with depth filtration. Viral filtration can be performed using micro- or nano-filters, such as those available from Asahi Kasei (Plavona®) and EDM Millipore (VPro®).

The term “polishing” is used herein to refer to one or more chromatographic steps performed to remove remaining contaminants and impurities such as DNA, host cell proteins, product-specific impurities, variant products and aggregates, and virus adsorption from a fluid including a recombinant multispecific protein that is close to a final desired purity. Polish chromatography makes use of resins and/or membranes containing agents that can be used in a flow-through mode (where the protein of interest flows through the resin/membrane and the contaminants and impurities are bound to the chromatography medium and the protein of interest is contained in the eluent), frontal or overloaded chromatography mode (where a solution containing the protein of interest is loaded onto a column until adsorption sites on are occupied and the species with the least affinity for the stationary phase (the protein of interest) starts to elute), or bind and elute mode (where the protein of interest is bound to the chromatography medium and eluted after the contaminants and impurities have flowed through or been washed off the chromatography medium). Examples of such chromatography methods include ion exchange chromatography (IEX), including anion exchange chromatography (AEX) and/or cation exchange chromatography (CEX); hydrophobic interaction chromatography (HIC); mixed modal or multimodal chromatography (MM), hydroxyapatite chromatography (HA); reverse phase chromatography, and gel filtration. In one embodiment the chromatographic method is cation exchange chromatography. In one embodiment, the cation exchange medium is a resin.

The invention provides a method of performing cation exchange chromatography under high salt loading conditions to reduce product-related impurities, the method comprising equilibrating a cation exchange chromatography medium with an equilibration buffer, loading the composition on to the cation exchange medium in a load buffer, washing the column with a first and second wash buffer, and eluting the multispecific protein from the cation exchange chromatography medium, wherein the equilibration, loading, and first wash buffer comprise 94-105 mM Sodium chloride.

“Cation exchange chromatography” refers to chromatography performed on a solid phase material that is negatively charged and has free cations for exchange with cations in an aqueous solution passed over or through the solid phase. The charge may be provided by attaching one or more charged ligands to the solid phase, e.g. by covalent linking. Alternatively, or in addition, the charge may be an inherent property of the solid phase (e.g. as is the case for silica, which has an overall negative charge). Commercially available cation exchange materials are available and include, but are not limited to, resin and membrane absorber medium, weak cation exchangers, strong cation exchangers, sulphopropyl (SP) immobilized on agarose (e.g. SP-SEPHAROSE FAST FLOW™, SP-SEPHAROSE FAST FLOW XL™ or SP-SEPHAROSE HIGH PERFORMANCE™, from GE Healthcare), CAPTO S™, CAPTO SP ImpRes™, CAPTO S ImpAct™ (GE Healthcare), FRACTOGEL-SO3™, FRACTOGEL-SE HICAP™, FRACTOPREP™ (EMD Merck), Fractogel® EMD 503-(M), Fractogel® EMD SE Hicap (M), Eshmuno® CPX, Eshmuno® S resins, Fractogel® EMD COO-(M), Mustang S Acrodisc with Mustang S AcroPrep with Mustang S, CM Ceramic HyperD® F AcroSep with CM Ceramic HyperD® F, among others.

For the inventive method, cation exchange chromatography is performed in bind and elute mode. An eluate or storage pool containing the multispecific protein of interest is loaded onto an equilibrated cation exchange medium such that the multispecific protein of interest is bound to the cation exchange medium. By “binding” the multispecific protein to the cation exchange material is meant exposing the multispecific protein to the cation exchange material under appropriate conditions (pH/conductivity) such that the multispecific protein is reversibly immobilized in or on the cation exchange material by virtue of ionic interactions between the multispecific protein of interest and a charged group or charged groups of the cation exchange material. The multispecific protein may be in an eluate or pool originating from a previous unit operation, such as affinity chromatography, neutralized low pH viral inactivation, depth filtration, or a polish chromatography operation.

The performance of cation exchange chromatography in bind and elute mode for the inventive method consists of several steps. In preparation for loading of the multispecific protein on to the cation exchange medium, the medium is equilibrated prior to loading with the same buffer composition as the multispecific protein composition. An “equilibration buffer” is the buffer used to equilibrate the chromatography material prior to loading the composition comprising a multispecific protein of interest.

Following equilibration, an eluate or storage pool containing a multispecific protein of interest from a previous unit operation is titrated with a high salt buffer formulation such at the final conditioned load buffer of the composition contains sodium chloride at a desired concentration. “Load buffer” and “final conditioned load buffer” are used interchangeably herein. The load buffer has a suitable formulation such that the multispecific protein of interest binds to the cation exchange material.

The loaded and bound cation exchange chromatography material is then subjected to a plurality of washes. Washing the cation exchange material means passing an appropriate wash buffer through or over the cation exchange material. The wash buffer removes one or more contaminants, including product-related impurities, from the cation exchange material, without substantial elution of the multispecific protein of interest. According to one embodiment of the invention there are two wash steps. In one embodiment there is a “first wash buffer” and a “second wash buffer”. The wash buffer is used to wash or re-equilibrate the cation exchange material prior to eluting the multispecific protein of interest. One or more of the wash buffer formulations may be the same as the equilibration and/or final conditioned load buffer formulations. The terms “first wash” and “second wash” should not be interpreted as excluding; the use of one or more additional washes or other buffers between the load and the first and/or second wash steps. Preferably by the end of the second wash step the UV baseline has returned to or is very near to zero, prior to the start of the elution.

The invention provides that the equilibration buffer, final conditioned load buffer, and at least one wash buffer has a high salt buffer formulation, in one embodiment, they all have the same high salt formulation buffer. In one embodiment the equilibration buffer, final conditioned load buffer, and at least one wash buffer comprises 94-105 mM Sodium chloride.

A “buffer” is a solution that resists changes in pH by the action of its acid-base conjugate components. In one embodiment the buffer is an acetate buffer. In one embodiment the buffer is a 100 mM acetate buffer. In one embodiment, the pH of the buffer is in the range of 5±0.05 to 5.0±0.1. In one embodiment, the range is pH 4.9 to 5.1. In one embodiment, the range is pH 4.95 to 5.05.

The equilibration buffer, final conditioned load buffer, and at least one wash buffer each also comprise a salt. In one embodiment, the salt is sodium chloride. In one embodiment the salt is sodium chloride in an amount from about 94 mM to about 105 mM.

In one embodiment the load buffer comprises 96-105 mM Sodium chloride. In one embodiment the load buffer comprises 94, 95, 96, 97, 98, 99, 100, 101, 102, 103, 104 or 105 mM Sodium chloride. In one embodiment the load buffer comprises 94 mM Sodium chloride. In one embodiment the load buffer comprises 96 mM Sodium chloride. In one embodiment the load buffer comprises 105 mM Sodium chloride.

In one embodiment of the invention, the load buffer comprises acetate. In one embodiment the load buffer comprises acetate, pH 4.9-5.1. In one embodiment the load buffer comprises acetate at pH, 4.9, 5.0, or 5.1. In one embodiment the load buffer comprises acetate, pH of 5.0±0.05 to 5.0±0.1. In one embodiment the load buffer comprises 100 mM acetate. In one embodiment the load buffer comprises 100 mM acetate, pH 4.9-5.1. In one embodiment the load buffer comprises 100 mM acetate at pH, 4.9, 5.0, or 5.1. In one embodiment the load buffer comprises 100 mM acetate, pH 5.0±0.05% to pH 5.0±0.1%.

In one embodiment the load buffer comprises acetate and 94 mM-105 mM Sodium chloride. In one embodiment the load buffer comprises acetate and 94 mM-96 mM Sodium chloride. In one embodiment the load buffer comprises acetate and 96 mM-105 mM Sodium chloride. In a related embodiment the load buffer comprises acetate and 94 mM Sodium chloride. In a related embodiment the load buffer comprises acetate and 96 mM Sodium chloride. In a related embodiment the load buffer comprises acetate and 105 mM Sodium chloride. In one embodiment the load buffer comprises acetate, 94 mM-105 mM Sodium chloride, pH 5.0±0.05 to 5.0±0.1. In a related embodiment the load buffer comprises acetate, 94-96 mM Sodium chloride, pH 5.0±0.05. In a related embodiment the load buffer comprises acetate, 96-105 mM Sodium chloride, pH 5.0±0.05. In a related embodiment the load buffer comprises acetate, 96 mM Sodium chloride, pH 5.0±0.05. In a related embodiment the load buffer comprises acetate, 105 mM Sodium chloride, pH 5.0±0.1. In a related embodiment the load buffer comprises acetate, 94 mM-105 mM Sodium chloride, pH 4.9-5.1. In a related embodiment the load buffer comprises acetate, 94 mM-105 mM Sodium chloride, pH of 4.9, 5.0 or 5.1. In a related embodiment the load buffer comprises acetate, 94 mM-105 mM Sodium chloride, pH of 5.0. In a related embodiment the load buffer comprises acetate, 94-96 mM Sodium chloride, pH of 4.9-5.1. In a related embodiment the load buffer comprises acetate, 96 mM Sodium chloride, pH of 5.0±0.05. In a related embodiment the load buffer comprises acetate, 105 mM Sodium chloride, pH of 5.0±0.1.

In one embodiment the load buffer comprises 100 mM acetate, 94 mM-105 mM Sodium chloride, pH 5.0±0.05 to 5.0±0.1. In a related embodiment the load buffer comprises 100 mM acetate, 94-96 mM Sodium chloride, pH 5.0±0.05. In a related embodiment the load buffer comprises 100 mM acetate, 96-105 mM Sodium chloride, pH 5.0±0.05. In a related embodiment the load buffer comprises 100 mM acetate, 96 mM Sodium chloride, pH 5.0±0.05. In a related embodiment the load buffer comprises 100 mM acetate, 105 mM Sodium chloride, pH 5.0±0.1. In a related embodiment the load buffer comprises 100 mM acetate, 94 mM-105 mM Sodium chloride, pH 4.9-5.1. In a related embodiment the load buffer comprises 100 mM acetate, 94 mM-105 mM Sodium chloride, pH of 4.9, 5.0 or 5.1. In a related embodiment the load buffer comprises 100 mM acetate, 94 mM-105 mM Sodium chloride, pH of 5.0. In a related embodiment the load buffer comprises 100 mM acetate, 94-96 mM Sodium chloride, pH of 4.9-5.1. In a related embodiment the load buffer comprises 100 mM acetate, 96 mM Sodium chloride, pH of 5.0±0.05. In a related embodiment the load buffer comprises 100 mM acetate, 105 mM Sodium chloride, pH of 5.0±0.1.

In one embodiment at least one wash buffer comprises 94-96 mM Sodium chloride. In one embodiment at least one wash buffer comprises 96-105 mM Sodium chloride. In one embodiment at least one wash buffer comprises 94, 95, 96, 97, 98, 99, 100, 101, 102, 103, 104 or 105 mM Sodium chloride. In one embodiment at least one wash buffer comprises 94 mM Sodium chloride. In one embodiment at least one wash buffer comprises 96 mM Sodium chloride. In one embodiment at least one wash buffer comprises 105 mM Sodium chloride.

In one embodiment of the invention, at least one wash buffer comprises acetate. In one embodiment at least one wash buffer comprises acetate, pH 4.9-5.1. In one embodiment at least one wash buffer comprises acetate at pH, 4.9, 5.0, or 5.1. In one embodiment at least one wash buffer comprises acetate, pH of 5.0±0.05 to 5.0±0.1. In one embodiment at least one wash buffer comprises 100 mM acetate. In one embodiment at least one wash buffer comprises 100 mM acetate, pH 4.9-5.1. In one embodiment at least one wash buffer comprises 100 mM acetate at pH, 4.9, 5.0, or 5.1. In one embodiment at least one wash buffer comprises 100 mM acetate, pH 5.0±0.05% to pH 5.0±0.1%.

In one embodiment at least one wash buffer comprises acetate, 94 mM-105 mM Sodium chloride. In one embodiment at least one wash buffer comprises acetate, 94 mM-96 mM Sodium chloride. In one embodiment at least one wash buffer comprises acetate, 96 mM-105 mM Sodium chloride. In a related embodiment at least one wash buffer comprises acetate, 94 mM Sodium chloride. In a related embodiment at least one wash buffer comprises acetate, 96 mM Sodium chloride. In a related embodiment at least one wash buffer comprises acetate, 105 mM Sodium chloride. In one embodiment at least one wash buffer comprises acetate, 94 mM-105 mM Sodium chloride, pH 5.0±0.05 to 5.0±0.1. In a related embodiment at least one wash buffer comprises acetate, 94-96 mM Sodium chloride, pH 5.0±0.05. In a related embodiment at least one wash buffer comprises acetate, 96-105 mM Sodium chloride, pH 5.0±0.05. In a related embodiment at least one wash buffer comprises acetate, 96 mM Sodium chloride, pH 5.0±0.05. In a related embodiment at least one wash buffer comprises acetate, 105 mM Sodium chloride, pH 5.0±0.1. In a related embodiment at least one wash buffer comprises acetate, 94 mM-105 mM Sodium chloride, pH 4.9-5.1. In a related embodiment at least one wash buffer comprises acetate, 94 mM-105 mM Sodium chloride, pH of 4.9, 5.0 or 5.1. In a related embodiment at least one wash buffer comprises acetate, 94 mM-105 mM Sodium chloride, pH of 5.0. In a related embodiment at least one wash buffer comprises acetate, 94-96 mM Sodium chloride, pH of 4.9-5.1. In a related embodiment at least one wash buffer comprises acetate, 96 mM Sodium chloride, pH of 5.0±0.05. In a related embodiment at least one wash buffer comprises acetate, 105 mM Sodium chloride, pH of 5.0±0.1.

In one embodiment at least one wash buffer comprises 100 mM acetate, 94 mM-105 mM Sodium chloride, pH 5.0±0.05 to 5.0±0.1. In a related embodiment at least one wash buffer comprises 100 mM acetate, 94-96 mM Sodium chloride, pH 5.0±0.05. In a related embodiment at least one wash buffer comprises 100 mM acetate, 96-105 mM Sodium chloride, pH 5.0±0.05. In a related embodiment at least one wash buffer comprises 100 mM acetate, 96 mM Sodium chloride, pH 5.0±0.05. In a related embodiment at least one wash buffer comprises 100 mM acetate, 105 mM Sodium chloride, pH 5.0±0.1. In a related embodiment at least one wash buffer comprises 100 mM acetate, 94 mM-105 mM Sodium chloride, pH 4.9-5.1. In a related embodiment at least one wash buffer comprises 100 mM acetate, 94 mM-105 mM Sodium chloride, pH of 4.9, 5.0 or 5.1. In a related embodiment at least one wash buffer comprises 100 mM acetate, 94 mM-105 mM Sodium chloride, pH of 5.0. In a related embodiment at least one wash buffer comprises 100 mM acetate, 94-96 mM Sodium chloride, pH of 4.9-5.1. In a related embodiment at least one wash buffer comprises 100 mM acetate, 96 mM Sodium chloride, pH of 5.0±0.05. In a related embodiment at least one wash buffer comprises 100 mM acetate, 105 mM Sodium chloride, pH of 5.0±0.1.

In one embodiment there is at least one additional wash step with a different wash buffer. In one embodiment at least one additional wash is a second wash. In one embodiment at least one additional wash buffer comprises 0-26 mM Sodium chloride. In one embodiment at least one additional wash buffer comprises 23-26 mM Sodium chloride. In one embodiment at least one additional wash buffer comprises 23, 24, 25, or 26 mM Sodium chloride. In one embodiment at least one additional wash buffer comprises 23 mM Sodium chloride. In one embodiment at least one additional wash buffer comprises 24 mM Sodium chloride. In one embodiment at least one additional wash buffer comprises 25 mM Sodium chloride. In one embodiment at least one additional wash buffer comprises 26 mM Sodium chloride. In one embodiment at least one additional wash buffer comprising 25 mM sodium chloride, pH of 5.0±0.05. In one embodiment at least one wash buffer comprises acetate and sodium chloride followed by at least one additional wash. In one embodiment least one wash buffer comprises acetate, 94-105 mM sodium chloride, followed by at least one additional wash. In one embodiment least one wash buffer comprises acetate, 94-105 mM sodium chloride, followed by at least one additional wash buffer comprising 0-26 mM Sodium chloride. In one embodiment the additional wash is a second wash. In a related embodiment the second wash buffer comprises 0-26 mM Sodium chloride. In one embodiment least one wash buffer comprises acetate, 94-96 mM sodium chloride, followed by at least one additional wash buffer comprising acetate, 25 mM sodium chloride. In one embodiment at least one wash buffer comprises acetate, 105 mM sodium chloride, followed by at least one additional wash buffer comprising acetate.

In one embodiment least one wash buffer comprises 100 mM acetate, 94-105 mM sodium chloride, pH of 5.0±0.05, followed by at least one additional wash. In a related embodiment the additional wash is a second wash. In a related embodiment the second wash buffer comprises 0-26 mM Sodium chloride. In one embodiment least one wash buffer comprises 100 mM acetate, 94-96 mM sodium chloride, pH of 5.0±0.05, followed by at least one additional wash buffer comprising 100 mM acetate, 25 mM sodium chloride, pH of 5.0±0.05. In one embodiment at least one wash buffer comprises 100 mM acetate, 105 mM sodium chloride, pH of 5.0±0.1, followed by at least one additional wash buffer comprising 100 mM acetate, pH of 5.0±0.1.

The bound multispecific protein is then eluted from the cation exchange chromatography material. The multispecific protein may be eluted by a gradient. The multispecific protein may be eluted from the cation exchange material by a linear or step gradient. Preferably the gradient is a salt gradient. The gradient is created using at least two elution buffers, wherein the combination of the buffers has a substantially increased conductivity such that the multispecific protein of interest is eluted from the cation exchange material. Preferably, the conductivity of the gradient is greater than that of the equilibrium and of each of the preceding buffers.

The cation exchange chromatography eluate can be subjected to further polish chromatography purification operations. Preferably at least one additional polish chromatography operation. Preferably the multispecific protein of interest is applied to the chromatography material in flow-through mode.

Concentration of the purified multispecific protein and buffer exchange into a desired formulation buffer for bulk storage of the drug substance can be accomplished by an ultrafiltration and diafiltration operation. Viral filtration can also be performed at any time during the downstream process.

Critical attributes and performance parameters of the purified multispecific protein can be measured to better inform decisions regarding performance of each step during manufacture. These critical attributes and parameters can be monitored real-time, near real-time, and/or after the fact. Key critical parameters such as media components that are consumed (such as glucose), levels of metabolic by-products (such as lactate and ammonia) that accumulate, as well as those related to cell maintenance and survival, such as dissolved oxygen content can be measured during cell culture. Critical attributes such as specific productivity, viable cell density, pH, osmolality, appearance, color, aggregation, percent yield, titer, concentration, viability, activity, and the like may be monitored during appropriated stages in the manufacturing process. Monitoring and measurements can be done using known techniques and commercially available equipment.

The pharmaceutical compositions (solutions, suspensions or the like), may include one or more of the following: buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives; sterile diluents such as water for injection, saline solution, preferably physiological saline, Ringer's solution, isotonic sodium chloride, fixed oils such as synthetic mono- or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

While the terminology used in this application is standard within the art, definitions of certain terms are provided herein to assure clarity and definiteness to the meaning of the claims. Units, prefixes, and symbols may be denoted in their SI accepted form. Numeric ranges recited herein are inclusive of the numbers defining the range and include and are supportive of each integer within the defined range. The methods and techniques described herein are generally performed according to conventional methods well known in the art and as described in various general and more specific references that are cited and discussed throughout the present specification unless otherwise indicated. See, e.g., Sambrook et al. Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990). All documents, or portions of documents, cited in this application, including but not limited to patents, patent applications, articles, books, and treatises, are hereby expressly incorporated by reference. What is described in an embodiment of the invention can be combined with other embodiments of the invention.

The present invention is not to be limited in scope by the specific embodiments described herein that are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

### EXAMPLES

### Example 1 No Salt Load Bi-Specific #1

Neutralized Protein A pool containing a fully human bi-specific, engineered immunoglobulin (Bi-specific #1) in an acetate buffer was loaded onto an Eshmuno CP-FT® cation exchange chromatography resin (GE Healthcare Bio-Science, Marlborough, Mass.) under the conditions outlined in Table 1.

TABLE 1
Conditions for cation exchange chromatography
under low salt load conditions

| | Bi-specific #1 |
|---|---|
| Column Size | 20 ± 2 cm, 140 ± 10% linear velocity |
| and rate | (cm/hr) |
| Concentration | 25 |
| Loading level | |
| (g/L) | |
| Pre Equilibration | 500 mM Acetate 2.5M Sodium Chloride pH 4.9 |
| Equilibration | 100 mM Acetate pH 5.0 |
| Wash | 100 mM Acetate pH 5.0 |
| Elution Buffer A | 100 mM Acetate pH 5.0 |
| Elution Buffer B | 100 mM Acetate 500 mM Sodium Chloride pH 5.0 |
| Elution Gradient | 0 |
| Start, % B | |
| Elution Gradient | 70 |
| End, % B | |
| Elution Gradient | 44 |
| Length, CV | |
| Elution Salt | 8.0 |
| Gradient, mM/CV | |
| Column Volumes | 44 |
| Yield (from pooled | 73% |
| fractions) | |
| Impurities in the | 1.5% HMW |
| recovered eluate | 1.5% half mAb |
| (from pooled | 0.8 LC1/LC2 ratio |
| fractions) | |

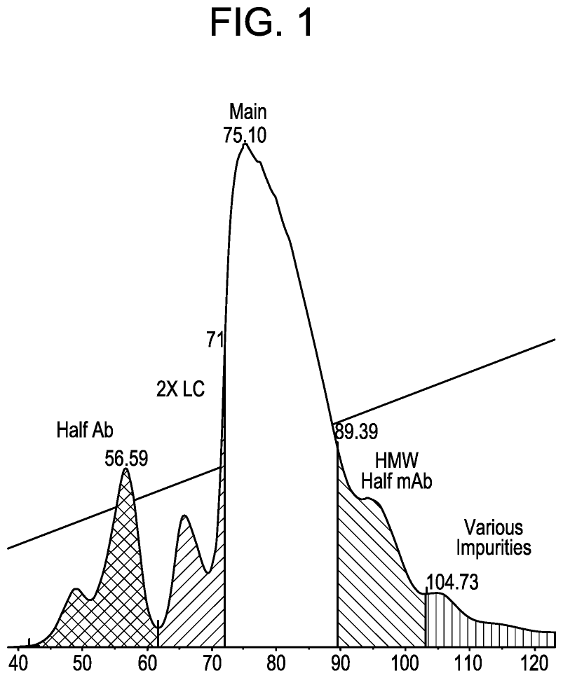

FIG. 1 shows that multiple impurities remained on the column and were eluted with the main product. These impurities were similar in charge (isoelectric point) to the main product and included half antibodies, 2× light chain-mis-assemblies and high molecular weight (HMW).

### Example 2 High Salt Load Conditioning Bi-Specific #1

A neutralized virus inactivated pool (100 mM Acetate, pH 5.0) containing Bi-specific #1 was combined with a load buffer (100 mM Acetate 350 mM sodium chloride pH 5.00) at a ratio of 1:0.378, giving a final conditioned load of 100 mM Acetate 96 mM Sodium Chloride pH 5.00. The conditioned load was loaded onto a Capto-SP ImpRes® cation exchange chromatography resin under the conditions described in Table 2.

TABLE 2
Conditions for cation exchange chromatography under high salt load conditions

| | Bi-specific #1 | | | |
|---|---|---|---|---|
| | CenterPoint pH | Lower pH and | Higher pH and | Lower pH, Salt, |
| | and Salt | Salt | Salt | and Load |
| Column Size and rate | 20 ± 2 cm, | 20 ± 2 cm, | 20 ± 2 cm, | 20 ± 2 cm, |
| | 140 ± 10% linear | 140 ± 10% linear | 140 ± 10% linear | 140 ± 10% linear |
| | velocity (cm/hr) | velocity (cm/hr) | velocity (cm/hr) | velocity (cm/hr) |
| Concentration | 25 | 23 | 27 | 15 |
| Loading level (g/L) | | | | |
| Pre-Equilibration | 500 mM Acetate | 500 mM Acetate | 500 mM Acetate | 500 mM Acetate |
| | 1.75 M Sodium | 1.75 M Sodium | 1.75 M Sodium | 1.75 M Sodium |
| | Chloride pH 4.9 | Chloride pH 4.9 | Chloride pH 4.9 | Chloride pH 4.9 |
| Equilibration | 100 mM Acetate | 100 mM Acetate | 100 mM Acetate | 100 mM Acetate |
| | 96 mM Sodium | 94 mM Sodium | 98 mM Sodium | 94 mM Sodium |
| | Chloride pH 5.00 | Chloride pH 4.95 | Chloride pH | Chloride pH |
| | | | 5.05 | 4.95 |
| Final Conditioned | 96 | 94 | 98 | 94 |
| Load [Sodium | | | | |
| chloride], mM | | | | |
| Wash 1 | 100 mM Acetate | 100 mM Acetate | 100 mM Acetate | 100 mM Acetate |
| | 96 mM Sodium | 94 mM Sodium | 98 mM Sodium | 94 mM Sodium |
| | Chloride pH 5.05 | Chloride pH 4.95 | Chloride pH | Chloride pH |
| | | | 5.05 | 4.95 |
| Wash 2 | 100 mM Acetate | 100 mM Acetate | 100 mM Acetate | 100 mM Acetate |
| | 25 mM Sodium | 23 mM Sodium | 26 mM Sodium | 24 mM Sodium |
| | Chloride pH 5.00 | Chloride pH 4.95 | Chloride pH | Chloride pH |
| | | | 5.00 | 4.95 |
| Elution Buffer A | 100 mM Acetate | 100 mM Acetate | 100 mM Acetate | 100 mM Acetate |
| | pH 5.00 | pH 4.95 | pH 5.05 | pH 4.95 |
| Elution Buffer B | 100 mM Acetate | 100 mM Acetate | 100 mM Acetate | 100 mM Acetate |
| | 350 mM Sodium | 350 mM Sodium | 350 mM Sodium | 350 mM Sodium |
| | Chloride pH 5.00 | Chloride pH 4.95 | Chloride pH | Chloride pH |
| | | | 5.05 | 4.95 |
| Elution Gradient | 7.1 | 7.0 | 7.3 | 7.0 |
| Start, % B | | | | |
| Elution Gradient | 100 | 100 | 100 | 100 |
| End, % B | | | | |
| Elution Gradient | 20.3 | 20.8 | 19.8 | 20.8 |
| Length, CV | 25-350 mM | 24-350 mM | 26-350 mM | 24-350 mM |
| | Sodium chloride | Sodium chloride | Sodium chloride | Sodium chloride |
| Elution Salt | 16.0 | 15.7 | 16.4 | 15.7 |
| Gradient, mM/CV | | | | |
| Column Volumes | 20.3 | 20.8 | 19.8 | 20.8 |
| CVs | | | | |
| Yield | 66% | 66% | 65% | 57% |
| Pool Volume, CVs | 4.1 | 3.3 | 4.6 | 1.9 |
| Impurities in the | HMW 0.9% | HMW 0.9% | HMW 0.9% | HMW 0.6% |
| recovered eluate | Half Mab 0.6% | Half Mab 0.5% | Half Mab 0.6% | |
| | LC1/LC2 ratio | LC1/LC2 ratio | LC1/LC2 ratio | |
| | 1.0% | 1.0% | 1.0% | |

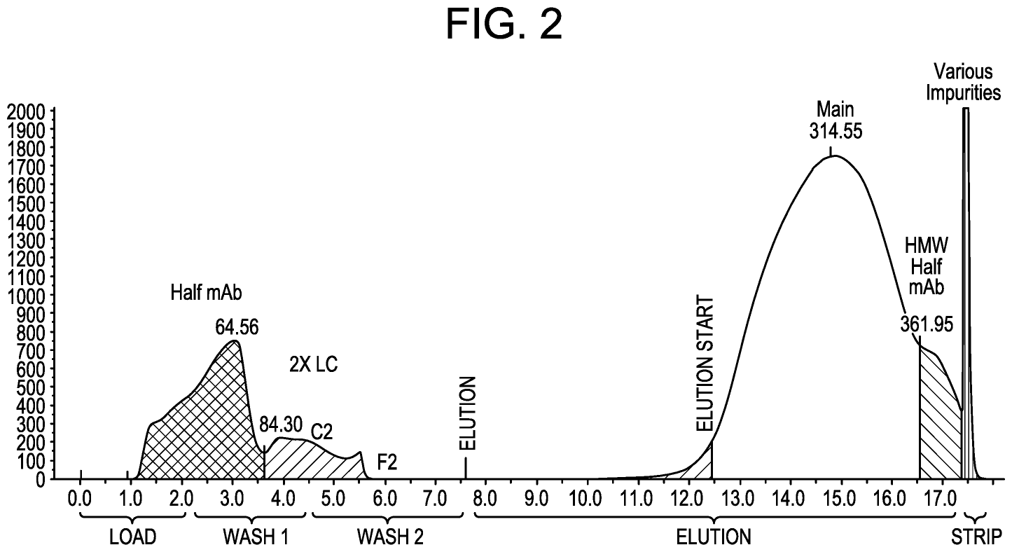

It was found that when high salt loading conditions were used, the lower pI impurities were removed from the resin before elution. FIG. 2 shows that high salt loading conditions resulted in a reduction in the number of impurity peaks in the elution profile, from four peaks to a single peak (shows CenterPoint results). The majority of the impurities were removed between the load and second wash steps. The half mAbs flowed through the resin during the load or were minimally bound to the column. The 2× LCs were removed from the column or minimally bound to the column following the first wash step. The second wash step provided complete binding conditions for the remaining protein species and reestablished the UV baseline to zero before the start of the elution, tightening the elution profile, resulting in a much more efficient collection and better quality of the main product.

The equilibration buffer, the final conditioned load, and the first wash buffer were tested at 94-98 mM sodium chloride, with similar results. The pH of the equilibration buffer, the final conditioned load, and the two wash buffer formulations were tested at various concentrations from 4.95 to 5.05, with similar results. The load concentration was tested at 5-27 mg/ml, with similar results.

Overall the high salt loads had a lower yield (˜60%) compared to the no salt load conditions. The reason is that with the no salt load run, the elution could be fractionated, allowing higher accuracy and precision in controlling the final product quality. However, the no salt load had a steep gradient of 8 mM[ Sodium chloride]/CV, which is not optimal for a robust manufacturing process, whereas the high salt loads had a salt gradient of 16 mM [Sodium chloride]/CV. The high salt load conditioning allowed for a reduction in the length of the elution from 44 column volumes to 20 column volumes. This reduction in column volume saves time and resources since the elution gradient is shorter, which also results in a more robust manufacturing process.

### Example 3 High Load Density, No Salt Load Conditioning, Bi-Specific #2

Neutralized Protein A eluate pool containing a fully human, engineered IgG/Fab fusion protein (Bi-specific #2), was loaded onto a Capto-SP ImpREs® cation exchange chromatography resin (GE Healthcare Bio-Science, Marlborough, Mass.) under the conditions outlined in Table 3.

TABLE 3
Conditions for high load density cation exchange
chromatography with no salt load conditioning

| | Bi-specific #2 |
|---|---|
| Column Size | 4.66 mL |
| and rate | 140 cm/h linear velocity |
| Concentration | 25 |
| Loading level | |
| (mg/ml) | |
| Pre Equilibration | 500 mM Acetate 1.75M Sodium Chloride pH 4.9 |
| Equilibration | 100 mM Acetate pH 5.0 |
| Wash | 100 mM Acetate pH 5.0 |
| Elution Buffer A | 100 mM Acetate pH 5.0 |
| Elution Buffer B | 100 mM Acetate 500 mM Sodium Chloride pH 5.0 |
| Elution Gradient | 0 |
| Start, % B | |
| Elution Gradient | 100 |
| End, % B | |
| Elution Gradient | 31 |
| Length, CV | |
| Elution Salt | 16 |
| Gradient, mM/CV | |
| Pool Column | 1.5 |
| Volumes | |
| (Fractions 4-6) | |
| Yield | 44% |
| Impurities in the | Reduced CE-SDS LC1/LC2 ratio = 0.9 |
| recovered eluate | SE HMW (%) = 1.7 |
| (corresponding to | SE LMW (%) = 0.9 |
| Fractions 4-6_{—} | SE-HPLC Monomer = 97.4% |

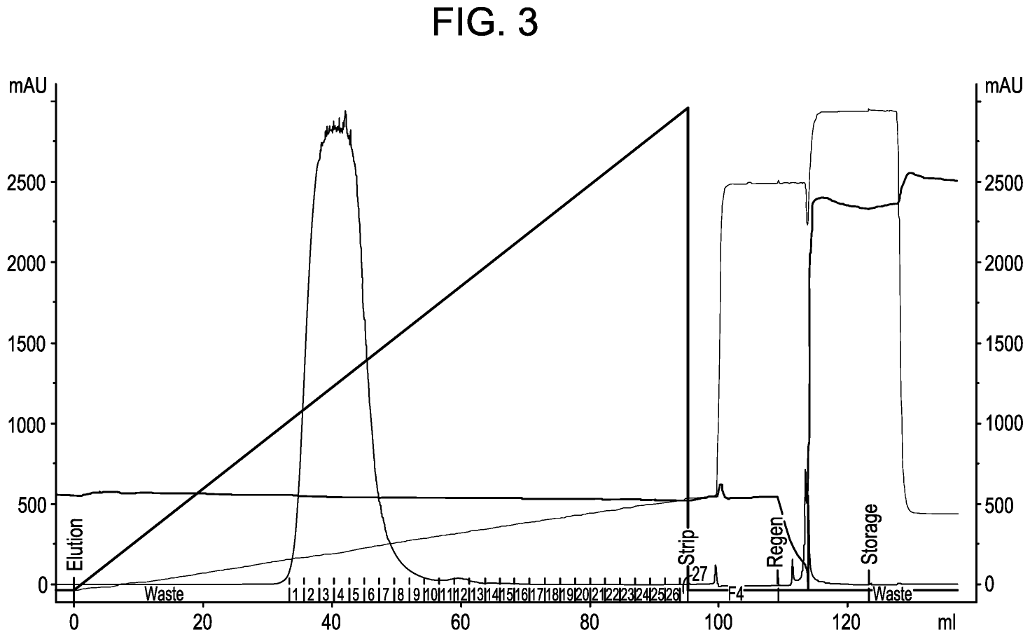

FIG. 3 shows one elution peak resulting from the high load density, no salt load conditioning, at a steep elution gradient. The low pI product-related impurities did not resolve from the main product under the high load density and are mostly in fractions 1, 2, and 3 as shown by a reduced CE-SDS LC1 to LC2 ratios of 4 to 7 (mispaired LC1 species) and LMW species of 2 to 4%.

### Example 4 Lower Load Density, No Salt Load Conditioning, Bi-Specific #2

The neutralized Protein A eluate pool containing the fully human, engineered IgG/Fab fusion protein (Bi-specific #2), was loaded onto a Capto-SP ImpREs® cation exchange chromatography resin (GE Healthcare Bio-Science, Marlborough, Mass.) under the conditions outlined in Table 4.

TABLE 4
Conditions for lower load density cation exchange
chromatography under no salt load conditions

| | Bi-specific #2 |
|---|---|
| Column Size | 4.66 mL |
| and rate | 140 cm/h linear velocity |
| Concentration | 10 |
| Loading level | |
| (mg/mL) | |
| Pre-Equilibration | 500 mM Acetate 1.75M Sodium Chloride pH 4.9 |
| Equilibration | 100 mM Acetate pH 5.0 |
| Wash | 100 mM Acetate pH 5.0 |
| Elution Buffer A | 100 mM Acetate pH 5.0 |
| Elution Buffer B | 100 mM Acetate 500 mM Sodium Chloride pH 5.0 |
| Elution Gradient | 0 |
| Start, % B | |
| Elution Gradient | 100 |
| End, % B | |
| Elution Gradient | 62 |
| Length, CV | |
| Elution Salt | 8 |
| Gradient, mM/CV | |
| Pool Column | 3.0 |
| Volumes | |
| Yield | 73% |
| Impurities in the | Reduced CE-SDS LC1/LC2 ratio = 1.2 |
| recovered eluate | SE HMW (%) = 1.4 |
| (corresponding to | SE LMW (%) = 0.1 |
| fractions 5-10) | SE-HPLC Monomer = 98.54% |

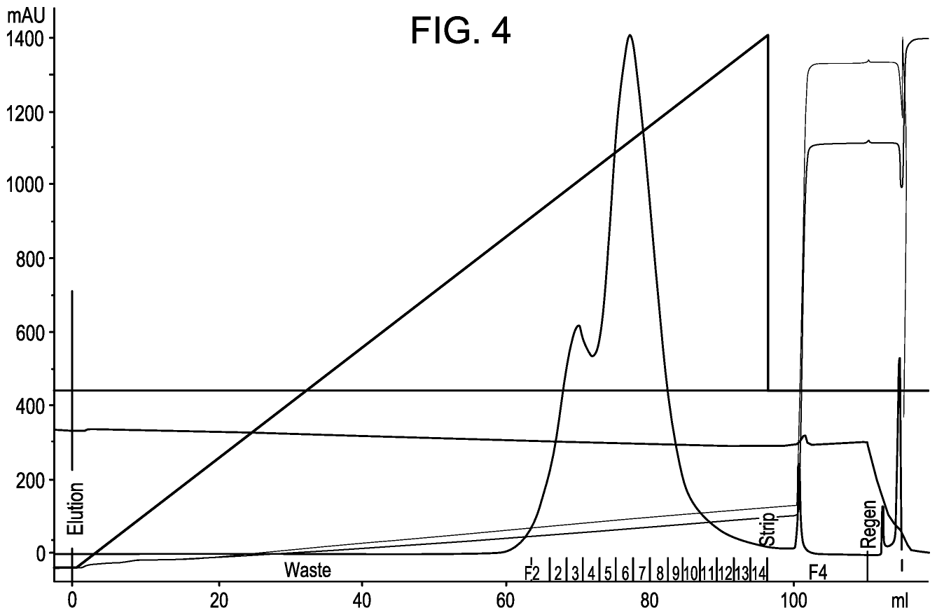

Since the high load density, steep elution gradient conditions of Example 3 did not provide sufficient resolution of the main product from the low pI product-related impurities, the load density and gradient conditions were reduced. FIG. 4 shows that a lower loading density (10 vs 25 g/L) and shallower gradient (8 vs 16 mM/CV) allowed for separation of the main low pI product-impurities into a distinct peak formed by fractions 1 to 4. This fraction showed a LC1 to LC2 ratio of 3 to 10, indicating mispaired LC1 species. In contrast, the main peak showed a cumulative LC1 to LC2 ratio of 1.2. While the resolution was better and increased the yield from 44% to 73%, because it still required automatic pooling based on OD, it would still be necessary to collect the eluate by starting above the highest OD for the pre-peak, lowering the yield, making this process insufficient for use in a manufacturing operation.

### Example 5 High Salt Load Conditioning, Bi-Specific #2

Neutralized virus inactivated pool containing Bi-specific #2 was combined with a high salt load buffer (100 mM Acetate 500 mM Sodium Chloride pH 5.00) giving a final conditioned load buffer concentration of 100 mM Acetate 105 mM Sodium Chloride pH 5.00. The conditioned load was loaded onto a Capto-SP ImpREs® cation exchange chromatography resin (GE Healthcare Bio-Science, Marlborough, Mass.) under the conditions outlined in Table 4.

TABLE 5
Conditions for cation exchange chromatography
under high salt load.

| | Bi-specific #2 |
|---|---|
| Column Size | 4.66 mL |
| and rate | 140 cm/h linear velocity |
| Concentration | 10 |
| Loading level | |
| (g/L) | |
| Pre Equilibration | 500 mM Acetate 1.75M Sodium Chloride pH 4.9 |
| Equilibration | 100 mM Acetate 105 mM Sodium Chloride pH 5.0 |
| Final Conditioned | 105 |
| Load [Sodium | |
| chloride], mM | |
| Wash 1 | 100 mM Acetate 105 mM Sodium Chloride pH 5.0 |
| Wash 2 | 100 mM Acetate pH 5.0 |
| Elution Buffer A | 100 mM Acetate pH 5.0 |
| Elution Buffer B | 100 mM Acetate 500 mM Sodium Chloride pH 5.0 |
| Elution Gradient | 0 |
| Start, % B | |
| Elution Gradient | 100 |
| End, % B | |
| Elution Gradient | 62 |
| Length, CV | |
| Elution Salt | 8.1 |
| Gradient, mM/CV | |
| Pool Column | 3.5 |
| Volumes (Fractions | |
| 5-10) | |
| Yield | 58% |
| Impurities in the | Reduced CE-SDS LC1/LC2 ratio = 1.1 |
| recovered eluate | SE HMW (%) = 1.0 |
| (corresponding to | SE LMW (%) = 0.1 |
| Fractions 4-10) | SE-HPLC Monomer = 98.9% |

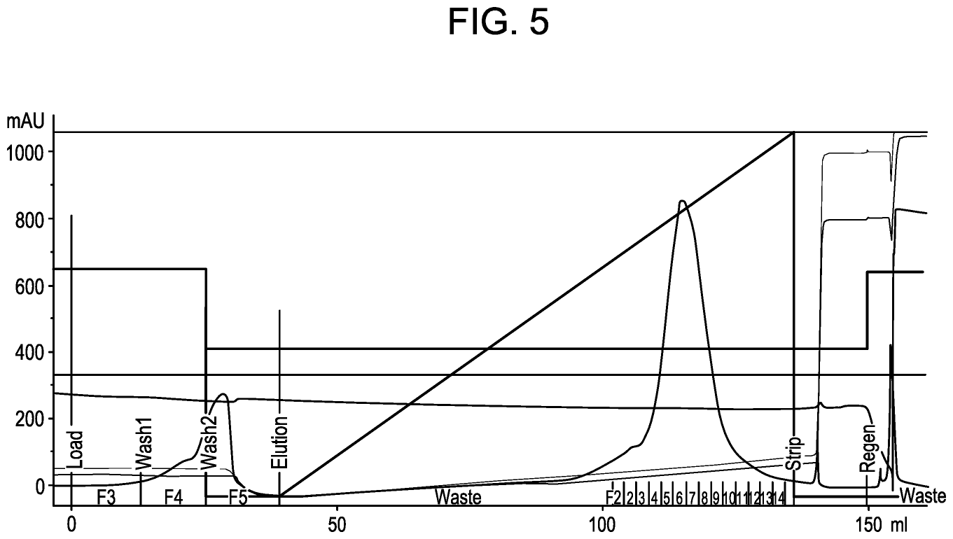

It was found that when high salt loading conditions were used, the low pI product-related impurities were removed from the CEX column before elution. These impurities likely correspond to mispaired LC1 species given that the LC1 to LC2 ratio on the collected wash 1 and wash 2 was 5.0, as compared to the expected ratio of 1 when the LC1 and LC2 correctly assemble and pair. FIG. 5 shows that under high salt loading conditions, there was a reduction in the number of impurity peaks in the elution profile from two peaks to a single peak with a small shoulder (Fractions 1-3) that still contained mispaired species (LC1/LC2=2 to 3). The second wash step also reestablished the UV baseline to zero before the start of the elution tightening the elution profile, resulting in a much more efficient collection and better quality of the main product. The optimized procedure with lower load level and shallower gradient, combined with a high salt conditioned load allowed an increased CEX purification yield from 44% to 58%, while enabling an elution profile for collecting a purified pool with low levels of mispaired LC1 species (as evidence by the LC1 to LC2 ratio close to 1), HMW and LMW, and an absorbance-based pooling criteria.

What is claimed is:

1. A method of purifying a multispecific protein from a composition comprising the multispecific protein and at least one product-related impurity, the method comprising
equilibrating a cation exchange chromatography medium with an equilibration buffer comprising 94-105 mM sodium chloride;
loading the composition on to the cation exchange medium in a load buffer comprises 94-105 mM sodium chloride;
washing the column with at least one wash buffer comprising 94-105 mM sodium chloride; and
eluting the multispecific protein from the cation exchange chromatography medium.

2. The method according to claim 1, wherein the load buffer comprises 94-96 mM sodium chloride.

3. The method according to claim 2, wherein the load buffer comprises 96-105 mM sodium chloride.

4. The method according to claim 2, wherein the load buffer comprises 94 mM sodium chloride.

5. The method according to claim 2, wherein the load buffer comprises 96 mM sodium chloride.

6. The method according to claim 2, wherein the load buffer comprises 98 mM sodium chloride.

7. The method according to claim 2, wherein the load buffer comprises 105 mM sodium chloride.

8. The method according to claim 1, wherein the load buffer comprises acetate.

9. The method according to claim 8, wherein the load buffer comprises acetate, pH 4.9-5.1.

10. The method according to claim 8, wherein the load buffer comprises acetate, pH 5.0±0.05 to 5.0±0.1.

11. The method according to claim 8, wherein the load buffer comprises 100 mM acetate.

12. The method according to claim 1, wherein the load buffer comprises acetate, 94-105 mM sodium chloride.

13. The method according to claim 1, wherein at least one wash buffer comprises 94-105 mM sodium chloride.

14. The method according to claim 13, wherein at least one wash buffer comprises 94-96 mM sodium chloride.

15. The method according to claim 13, wherein at least one wash buffer comprises 96-105 mM sodium chloride.

16. The method according to claim 13, wherein at least one wash buffer comprises 94 mM sodium chloride.

17. The method according to claim 13, wherein at least one wash buffer comprises 96 mM sodium chloride.

18. The method according to claim 13, wherein at least one wash buffer comprises 98 mM sodium chloride.

19. The method according to claim 13, wherein at least one wash buffer comprises 105 mM sodium chloride.

20. The method according to claim 1, wherein at least one wash buffer comprises acetate.

21. The method according to claim 20, wherein at least one wash buffer comprises acetate, pH 4.9-5.1.

22. The method according to claim 20, wherein at least one wash buffer comprises acetate, pH 5.0±0.05 to 5.0±0.1.

23. The method according to claim 20, wherein at least one wash buffer comprises 100 mM acetate.

24. The method according to claim 1, wherein at least one wash buffer comprises acetate, 94 mM-105 mM sodium chloride.

25. The method according to claim 1, wherein the method comprises at least one additional wash buffer.

26. The method according to claim 25, wherein at least one additional wash buffer is a second wash buffer.

27. The method according to claim 25, wherein at least one additional wash buffer comprises 0-26 mM sodium chloride.

28. The method according to claim 25, wherein at least one wash buffer comprises acetate, 94-105 mM sodium chloride, followed by at least one additional wash buffer comprising acetate, 0-25 mM sodium chloride.

29. The method according to claim 1, wherein at least one equilibration buffer comprises 94-105 mM sodium chloride.

30. The method according to claim 29, wherein at least one equilibration buffer comprises 94-96 mM sodium chloride.

31. The method according to claim 29, wherein at least one equilibration buffer comprises 96-105 mM sodium chloride.

32. The method according to claim 29, wherein at least one equilibration buffer comprises 94 mM sodium chloride.

33. The method according to claim 29, wherein at least one equilibration buffer comprises 96 mM sodium chloride.

34. The method according to claim 29, wherein at least one equilibration buffer comprises 98 mM sodium chloride.

35. The method according to claim 29, wherein at least one equilibration buffer comprises 105 mM sodium chloride.

36. The method according to claim 1, wherein the equilibration buffer comprises acetate.

37. The method according to claim 36, wherein the equilibration buffer comprises acetate, pH 4.9-5.1.

38. The method according to claim 36, wherein the equilibration buffer comprises acetate, pH 5.0±0.05 to 5.0±0.1.

39. The method according to claim 36, wherein the equilibration buffer comprises 100 mM acetate.

40. The method according to claim 1, wherein the composition is loaded at 10-27 g/L.

41. The method according to claim 40, wherein the composition is loaded at 15-27 g/L.

42. The method according to claim 1, wherein the multispecific protein is eluted from the cation exchange resin by a gradient.

43. The method according to claim 42, wherein the gradient is linear.

44. The method according to claim 42, wherein the gradient is a salt gradient.

45. The method according to claim 1, wherein the multispecific protein is a bispecific protein.

46. The method according to claim 1, wherein the multispecific protein is a bispecific antibody.

47. A purified, multispecific protein prepared by a method according to claim 1.

48. The method according to claim 1, wherein the cation exchange chromatography medium is a resin.

49. A method of reducing low pI impurities in the eluate from cation exchange chromatography, the method comprising
equilibrating a cation exchange chromatography medium with an equilibration buffer comprising 94-105 mM Sodium chloride;
loading the composition on to the cation exchange medium in a load buffer comprises 94-105 mM Sodium chloride;
washing the column with at least one wash buffer comprising 94-105 mM Sodium chloride; and
eluting the multispecific protein from the cation exchange chromatography medium;
wherein the cation exchange chromatography eluate has reduced low pI impurities compared to the cation exchange chromatography eluate recovered in a corresponding method in which no sodium chloride is used in the equilibration, load, and wash steps.

50. The method according to claim 49, wherein the low pI impurity is a product-related impurity.

51. The method according to claim 50, wherein at least one product-related impurity is a half antibody or 2×, 3×, or 4× light chain-mis-assembly.

52. A method of performing cation exchange chromatography under high salt loading conditions to reduce product-related impurities, the method comprising
equilibrating a cation exchange chromatography medium with an equilibration buffer;
loading the composition on to the cation exchange medium in a load buffer;
washing the column with a first and a second wash buffer; and
eluting the multispecific protein from the cation exchange chromatography medium;
wherein the equilibration, loading and first wash buffers comprise 94-105 mM Sodium chloride.

53. The method according to claim 52, wherein the second wash buffer comprises 0-26 mM Sodium chloride.

54. A method of producing an isolated, purified, recombinant multispecific protein, the method comprising
establishing a cell culture in a bioreactor with a host cell expressing the multispecific protein;
culturing the host cells to express the multispecific protein;
harvesting the recombinant multispecific protein;
affinity purifying the harvested recombinant multispecific protein;
inactivating virus at low pH in the eluate pool from the affinity purification and neutralizing the pool;
equilibrating a cation exchange chromatography medium with an equilibration buffer comprising 94-105 mM Sodium chloride;
loading the neutralized affinity purified recombinant multispecific protein on to the equilibrated cation exchange medium in a load buffer comprises 94-105 mM Sodium chloride;
washing the cation exchange medium with a wash buffer comprising 94-105 mM Sodium chloride, followed by a second wash buffer comprising 0-26 mM Sodium chloride;
eluting the multispecific protein from the cation exchange chromatography medium;
loading the cation exchange chromatography eluate comprising the recombinant multispecific protein onto a second chromatography resin in flow through mode; and
concentrating the purified recombinant multispecific protein in a formulation buffer.

55. The method according to claim 47, wherein the second chromatography resin is selected from an anion exchange chromatography resin, cation exchange chromatography resin, multi-modal chromatography resin, hydrophobic interaction chromatography resin, and hydroxyapatite chromatography resin.

56. An isolated, purified, recombinant multispecific protein prepared by a method according to claim 55.

57. A pharmaceutical composition comprising the isolated, purified, recombinant multispecific protein prepared by a method according to claim 55.
